(19) United States
(12) Patent Application Publication
(10) Pub. No.: US 2026/0263830 A1
(43) Pub. Date: Sep. 10, 2026

(54) OPTOGENETIC MODULATION OF CENTRAL NERVOUS SYSTEM FOR TREATING PAIN

(71) Applicant: Opsin Biotherapeutics, Inc., Bedford, TX (US)

(72) Inventors: Samarendra Kumar Mohanty, Bedford, TX (US); Darryl Narcisse, Bedford, TX (US); Robert BENKOWSKI, Bedford, TX (US)

(21) Appl. No.: 19/157,653

(22) PCT Filed: Feb. 20, 2024
(86) PCT No.: PCT/US2024/016566

§ 371 Date: Aug. 18, 2025

Related U.S. Application Data
(60) Provisional application No. 63/447,029, filed on Feb. 20, 2023.

(51) Int. Cl. A61N 5/06; A61K 38/17; A61K 41/00; A61K 48/00; A61P 29/02

(52) U.S. Cl. CPC A61N 5/062; A61K 38/177; A61K 41/0057; A61K 48/005; A61N 5/0601; A61P 29/02; A61N 2005/063; A61N 2005/0663

(57) ABSTRACT

This invention relates generally to compositions and methods for modulating cellular activities using multi-characteristic opsin (MCO). Further, the invention provides methods for treating ailments related to activity of the dorsal root ganglia (DRG), anterior cingulate cortex (ACC) or spinal cord including neuropathic pain. Specific embodiments include methods of modulating inhibitory pathways to inhibit chronic neuropathic pain. The methods can use a light sensitive optogenetic actuator with inhibitory neuron specificity. Embodiments also include an optogenetic pain modulator device that can regulate the frequency and intensity of optogenetic stimulation.

### RELATED APPLICATIONS

This application claims priority to U.S. provisional patent application No. 63/447,029, filed Feb. 20, 2023, the contents which are incorporated herein by reference.

### REFERENCE TO SEQUENCE LISTING

The entire content of the following electronic submission of the sequence listing via the USPTO Patent Center, as set forth in MPEP § 173011.B.2(a)(C), is hereby incorporated by reference in its entirety for all purposes. The sequence listing is identified on the electronically filed text file as follows: File Name: OB1-003WO; Date of Creation: Feb. 20, 2024; Size (bytes): 5 KB.

### FIELD OF THE INVENTION

The invention relates to methods of therapeutic methods, and more specifically, it relates to a method of reducing pain using an optogenetic actuator to activate inhibitory neurons of the dorsal root ganglia (DRG), anterior cingulate cortex (ACC) or spinal cord.

### BACKGROUND

Pain can be defined as an unpleasant sensory and emotional experience associated with or resembling that associated with actual or potential tissue damage. In the field of medicine, pain is regarded as a symptom of an underlying condition. The sensation of pain can provide important information about the status of the body or any current injury as well as motivating behavior that is more conducive to the preservation of bodily integrity. However, due to dysfunction or unresolved disease pathology, pain itself can become a debilitating problem.

Pain can be characterized based on different criteria. For example, it can be described by location, duration, mechanism and underlying cause. Describing pain by location or cause (often malignant or non-malignant) is relatively self-explanatory. The main mechanisms by which pain is experienced is nociceptive (i.e., pain from pain nerves reporting information) and neuropathic (i.e., pain from dysfunction of the pain nerves or related neurons themselves). Pain can also be classified by the duration of pain. Most pain experiences are acute (e.g., nociceptive pain related directly to an injury and dissipating over time with healing or acclimation). However, when pain persists for longer than three to six months (i.e., longer than the normal healing process), it becomes chronic pain. Its constant nature and persistence negate the warning benefit of pain and cause it to become a debilitating chronic condition in need of treatment itself. When left untreated long enough, acute pain can cause secondary changes in the central nervous system that result in chronic pain syndromes.

Chronic pain is a common condition affecting an estimated 15-20% of the global adult population. It is estimated that debilitating chronic pain costs $560 to $635 billion annually and affects approximately 100 million Americans. The economic costs of debilitating chronic pain are also further complicated by the unintended effects of common treatments. Opioids are a common effective treatment for pain but are associated with dose-limiting adverse effects such as respiratory depression and addiction. Given the nature of chronic pain, treatment must be as constant as the pain itself. According to the CDC, the probability of addiction increases as the duration of treatment increases. Even one-day prescriptions can carry a 2.9% risk of addiction, increasing to a 30% risk with month-long prescriptions.

Non-opioid drugs for pain treatment have been developed as alternatives to opioids. For example, anti-epileptics are a common class of drugs used for the treatment of neuropathic pain. However, they can induce serious central nervous system-related side effects including drowsiness, somnolence, loss of consciousness as well as cardiac side effects. Non-medicament methods for pain treatment have been pursued for pain treatment. They include spinal cord stimulation, peripheral nerve stimulation, and deep brain stimulation by electrical methods. These methods have similar limitations as opioids in that they are nonspecific in their interaction with human biology. For example, electrical discharges activate any neurons within a specific area of effect thus leading to unintended consequences including speech deficits, personality changes, and possibly cognition.

Therefore, developing effective pain management, it is necessary to determine which anatomical structures and cell types are involved in the detection, transmission, processing, experience, and consequences of pain (making them viable treatment targets) as well as alternate ways to modulate these treatment targets for pain modulation. Efforts are currently underway to develop better methods and drugs that are devoid of serious side effects to provide more significant pain relief to a broader patient population.

Previous research has established the major nerve types that detect pain. However, they are localized, and thus targeted delivery of pain treatment to those areas can result in numbness and fail to address more widespread injury/dysfunction or diffuse pain states such as visceral pain. Moreover, central pain conditions such as the dysfunctional changes that result in chronic pain syndrome are entirely unaddressed by this treatment strategy. Therefore, the targeting of higher-level neurons in the central nervous system (CNS) is warranted. The role of specific structures of the CNS in the pain experience is a much more complex issue as the study of this anatomy is complicated by its sensitivity and essential role in life as well as the interconnectedness of various substructures for the formation of a functional CNS. Despite this, multiple studies have established the role of major structures in pain such as the Dorsal Root Ganglia (DRG) in the Spinal cord, the Thalamus (for routing pain information, the insula, and the Anterior Cingulate Cortex (ACC). Multiple case reports and experimental studies in both animals and humans suggest that neurons of the ACC are involved in pain perception (among the many other functions of the ACC), specifically the affective-aversive aspects of the pain experience.

Another complicating factor in pain drug discovery relates to differences in types of pain. Pain pathologies can be diverse and different pain conditions will likely require a variety of tailored therapeutic agents. For example, pain associated with post-herpetic neuralgia, diabetic painful neuropathy, and fibromyalgia may need three different types of drugs. It is therefore important to generate preclinical data that can reliably predict the specific action of new drugs on the human pain pathway and provide information as to which pain type may be best treated by a given drug candidate. In recent years, progress has been made in the field of clinical diagnostic neurology. Thanks to the introduction of a variety of tests conducted on patients, it is possible to begin establishing with some accuracy the type of somatosensory nerve fibers affected by a painful pathology and, in some cases, even assess the extent and type of damage. This information can be valuable for guiding the selection of treatment options.

Because conventional treatments for pain are generally ineffective, there is a need for alternative methods to treat pain. Conventional drugs can be addictive and/or lead to numerous unwanted side effects. Non-medicament methods such as electrode implantation lack specificity and are generally ineffective in treating pain. Applicant has discovered methods of optogenetic modulation that overcome these limitations by allowing cell-specific stimulation with low power.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

Embodiments of the invention include a recombinant virus that includes the recombinant nucleic acids disclosed herein. In some embodiments, the virus is a recombinant adeno-associated virus (AAV). In aspects, a multi-characteristic opsin (MCO) of SEQ ID NO. 1 is expressed using the virus.

In aspects, the multi-characteristic opsin (MCO) expressed in the methods described herein has at least 80%, at least 85%, at least 90% or at least 95% sequence identity to SEQ ID NO. 1.

Embodiments also include a method of optically controlling neural activity in a cell. The method can include expressing in a cell a recombinant nucleic acid provided herein and controlling the neural activity of the cell with light to modulate the expression of the light-sensitive protein.

Embodiments also include a method of inhibiting pain in a subject. The method can include steps of (a) expressing a recombinant nucleic acid encoding a light-sensitive protein in cells of spinal cord cells, the DRG or ACC of the subject and (b) controlling expression of the light-sensitive protein to modulate the neural activity of the cells with light. The modulation can treat the pain by reducing the duration and/or intensity.

In aspects, the light/illumination comes with a low power burden and can be tuned to respond to wavelengths that propagate better through tissues without damaging cells.

Embodiments also include a method of inhibiting neuropathic pain without affecting nociceptive pain. The method can include steps of (a) expressing recombinant multi-characteristic opsin (MCO) in the neural tissue of the subject and (b) controlling the MCO with light to modulate the activity of the neural tissue. The expression of the MCO can treat neuropathic pain by reducing the duration and/or intensity.

In aspects, the neuropathic pain is caused by one or more of a traumatic insult, a spinal cord injury, a limb amputation, a contusion, an inflammation or a surgical procedure, an ischemic event, an infectious agent, exposure to a toxic agent or a disease.

Embodiments also include a method of treating an ailment in a subject. The method can include steps of (a) expressing a recombinant nucleic acid encoding a light-sensitive protein in cells of the neural tissue of the subject and (b) modulating the activity of the neural tissue with light that controls the expression of the light-sensitive protein. The modulation can adjust the excitation to inhibition (E/I) balance of the neural tissue to treat the ailment. The ailment can be, for example, pain and/or a neurological disorder.

In aspects, the neurological disorder is one or more of the fibromyalgia, rheumatoid arthritis, osteoarthritis, chronic arthropathy, spinal nerve compression syndromes associated with neoplasia and/or disc herniation, chronic back pain, chronic joint pain of any etiology associated with inflammation and/or structural joint abnormalities, post-herpetic neuralgia, trigeminal neuralgia, chronic metabolic neuropathy associated with chronic pain, migraine, inflammatory pain, post-surgical pain syndromes including phantom limb pain, post-traumatic stress disorder (PTSD), irritable bowel syndrome, autonomic neuropathies, arachnoiditis, chronic regional pain syndrome, vulvodynia, and chronic pain syndrome associated with activation of central sensitization pathways, visual impairment, drug addiction, a psychological disorder, and a movement disorder.

Embodiments also include a recombinant nucleic acid that includes: a nucleic acid fragment encoding a light-sensitive protein and a regulatory nucleic acid fragment that is capable of directing selective expression of the light-sensitive protein in a cell of the CNS. In embodiments, the light-sensitive protein is sensitive to a light that is a visible light or a light that is delivered transdermally. In some embodiments, the light-sensitive protein can modulate the neuronal activity of cells of the spinal cord, DRG and/or ACC, including activity related to pain transmission or generation.

In some embodiments, the light-sensitive protein is a membrane-bound microbial opsin. In some embodiments, the microbial opsin is a photosensitive ion channel or pump.

Embodiments also include an ambient light activatable multi-characteristic opsin (MCO) encoding gene, which is activated with light. In aspects, the MCO is packaged into a safe viral vector (e.g., AAV) with a fluorescent reporter (e.g., vMCO1-mCherry).

Embodiments also include an AAV vector carrying a red-light sensitive optogenetic actuator with inhibitory neuron specificity to target inhibitory (e.g., GABAergic) neurons of the DRG and/or ACC for treating an ailment and/or inhibiting pain responses.

Another embodiment is a method to relieve neuropathic pain. The method can include a step of optically modulating activity related to pain neurotransmission or generation. The method can include expressing in a cell of the subject the recombinant nucleic acid provided herein, and controlling the neural activity of the cell with light to modulate the expression of the light-sensitive protein, thereby relieving the neuropathic pain. The controlling can be implemented with high spatial and temporal precision using a specifically positioned device where the light emission is controlled over time.

Embodiments include a method of expressing in target cells of a subject a recombinant nucleic acid and controlling the neural activity of the cell with light to modulate the expression of a light-sensitive protein, thereby relieving neuropathic pain. The method can provide significant analgesia for chronic neuropathic pain without off-target effects, such as general central nervous system depression. In embodiments, the target cells (for expression of the recombinant protein) are cells of the spinal cord, DRG and/or ACC.

Further embodiments include methods of optogenetic stimulation that reduce inflammatory pain but do not affect the initial warning nociceptive pain that provides information about the localization and intensity of bodily damage.

In aspects, the methods described herein can altera the balance between excitation to inhibition (i.e., the E:I ratio) of neural cells.

The results described herein support modulation of the inhibitory pathways within the DRG and ACC as a viable alternative for inhibiting chronic neuropathic pain.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a flowchart of steps in a method of optogenetic stimulation.

FIG. 2A is an image of a wireless optogenetic stimulation device.

FIG. 2B is an image of an optical fiber of the device that emits low-power red light while being minimally invasive.

FIG. 2C is an image of mouse brain tissue showing the propagation of red light.

FIG. 2D is a graph depicting an intensiometric analysis of light propagation through a brain section.

FIG. 2E is a graph showing that the presence of the implant does not significantly affect acute pain responses in the formalin assay.

FIG. 2F is an image of a mouse showing that the presence of the implant does not significantly affect acute pain response as measured by licking.

FIG. 2G is an image of a mouse showing that the presence of the implant does not significantly affect acute pain response as measured by paw lifting.

FIG. 3A is an image showing intrinsic mCherry fluorescence imaging of GAD67 promoter-driven MCO expression in the anterior cingulate cortex (ACC) two weeks after pMCO₂ injection (optical fiber outline indicated by arrows).

FIG. 3B is an image of a wireless fiber-coupled red LED implanted in MCO-transfected mouse.

FIG. 3C is an image of a confocal immune stained mouse brain slice: DAPI (nuclear stain).

FIG. 3D is an image of a confocal immune stained mouse brain slice: GAD65-marker for GABAergic neurons.

FIG. 3E is an image of a confocal immunostained mouse brain slice: MCO Reporter-mCherry.

FIG. 3F is an image of a confocal immune stained mouse brain slice: Overlay of GAD65 & mCherry.

FIG. 3G is an image of a confocal immunostained mouse brain slice; Zoomed areas (marked by a rectangle in f) show colocalization of GAD65.

FIG. 3H is an image of a confocal immune stained mouse brain slice: Zoomed areas (marked by rectangle in f) showing colocalization of mCherry.

FIG. 4A is a flowchart showing the steps in assessing the effect of optogenetic modulation on acute pain model (formalin injected into the hind paw).

FIG. 4B is a graphical depiction of baseline formalin-induced pain scores at 10-minute intervals compared with that during continuous 5 Hz optogenetic stimulation (5 ms pulses at 630 nm).

FIG. 4C shows baseline formalin-induced pain scores 11 days after early transduction.

FIG. 4D shows the average of the cumulative pain scores (measured in 5-minute intervals) in the early (0-11 minutes) non-inflammatory and late (20-41 minutes) inflammatory phases of pain 11 days early post-transduction.

FIG. 4E shows the average of the cumulative pain scores five weeks post optimal transduction.

FIG. 5A is a graph showing baseline formalin-induced pain scores at 10-minute intervals compared with 5 Hz optogenetic stimulation (5 ms pulses at 630 nm) for 5-minute on-off intervals.

FIG. 5B shows baseline formalin-induced pain scores with 2 Hz optogenetic stimulation for five minute on-off intervals.

FIG. 5C shows baseline formalin-induced pain scores with 5 Hz optogenetic stimulation starting 15 minutes into the experimental session. The average of the cumulative pain scores (measured in 5-minute intervals) in the early (0-11 minutes) non-inflammatory and late (20-41 minutes) inflammatory phases of pain.

FIG. 5D shows baseline formalin-induced pain scores with intermittent 5 Hz optogenetic stimulation.

FIG. 5E shows baseline formalin induced pain scores with intermittent 2 Hz optogenetic stimulation.

FIG. 5F shows baseline formalin-induced pain scores with delayed 5 Hz optogenetic stimulation.

FIG. 6A is a flowchart depicting the steps of mechanical hypersensitivity chronic pain experimentation.

FIG. 6B is an image of Manual Von Frey apparatus showing the presentation of a Von Frey filament to the hind paw of a baseline mouse.

FIG. 6C is a chart showing the minimum force needed to elicit hind paw withdrawal response to mechanical stimulation increases with increased duration of exposure.

FIG. 6D is a chart showing the percent change in withdrawal force relative to the mouse's untreated baseline threshold.

FIG. 7A is a flow chart that shows the steps of chronic pain experimentation.

FIG. 7B is an image of a CPP apparatus showing the dark (no treatment) chamber (left), the central chamber, and the lit (treatment-associated) chamber (right).

FIG. 7C is a comparison of chamber preferences of naïve unimplanted and implanted mice in the CPP apparatus.

FIG. 7D is a chart showing the increased percentage of post-conditioning time spent in treatment.

FIG. 8A is a graphical depiction showing the change in temperature of the irradiated spot in live brain measured by infrared (IR) camera.

FIG. 8B is a set of images demonstrating no loss of viability of ACC-GABAergic neurons expressing MCO-mCherry after chronic optogenetic stimulation. i) DAPI; (ii) MCO-mCherry; (iii) Caspase-3; and (iv) Overlay of Bi-iii.

FIG. 8C is a set of images demonstrating that injection of AAV-MCOII led to MCO expression without causing an inflammatory response. (i) DAPI; (ii) Immunostained mCherry confirming MCO-expression; (iii) CD45 immunostain (green) absent in ACC regions injected with 3 ml of AAV-MCO (8×10¹² vg/ml). (iv) Overlay of Ci-iii.

FIG. 8D is a set of images demonstrating no immune cell response to implant in ACC transfected with MCO-mCherry. (i) DAPI; (ii) MCO-mCherry; (iii) Iba1 (marker for microglia/macrophages); and (iv) Overlay of Di-iii. Minimal Iba1 +ve (green) cells observed in the vicinity of the implant.

FIG. 9A is a graphical depiction of formalin assay scores at ten-minute intervals.

FIG. 9B is a graphical depiction of average pain score over time which shows that pain responses are reduced even when delayed.

FIG. 10A is a graph of force to cause hyperalgesic paw withdrawal over time which shows the modulatory effect of optogenetic stimulation.

FIG. 10B is a graph that shows the percentage of mice that retained an increased sensitivity threshold.

FIG. 11A is an image of a three-chamber social novelty apparatus.

FIG. 11B is a graphical depiction of the results of a social impact study which shows that MCO transfection does not affect social interaction.

FIG. 12A is a graphical depiction of average pain scores over time which shows that acute pain responses are reduced with continuous 5 Hz treatment.

FIG. 12B is a bar graph of force needed for paw withdrawal in different groups of mice.

FIG. 13A is an image of a Y-maze apparatus for studying social behavior.

FIG. 13B is a bar graph that shows the percentage of alternations in treated and untreated mice.

FIG. 14A is a bar graph that shows the results of formalin assay scores at ten-minute intervals with 5 ms pulses.

FIG. 14B is a bar graph that shows the results of formalin assay scores and compares the baseline to treated experimental mice and mice without (i.e., aborted) treatment.

FIG. 15A is a set of images showing the results of intrathecal delivery to an axial section of a spinal cord and associated nerves.

FIG. 15B is a set of images showing the results of paraspinal delivery to an axial section of a spinal cord and associated nerves.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification do not necessarily all refer to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods, and their related results according to the embodiments of the present disclosure are given below. Note that titles or subtitles may be used in the examples for the convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “BLUETOOTH™” refers to a short-range wireless technology standard that is used for exchanging data between fixed and mobile devices over short distances using UHF radio waves in the ISM bands, from 2.402 to 2.48 GHz, and building personal area networks.

The term “neural cell” refers to a type of cell that receives and sends messages from the body to the brain and back to the body. Within a nervous system, a neuron or nerve cell is an electrically excitable cell that fires electric signals called action potentials across a neural network. Neurons communicate with other cells via synapses, which are specialized connections that commonly use minute amounts of chemical neurotransmitters to pass the electric signal from the presynaptic neuron to the target cell through the synaptic gap. Neurons are the main components of nervous tissue in all animals except sponges and placozoa. Neurons are typically classified into three types based on their function. Sensory neurons respond to stimuli such as touch, sound, or light that affect the cells of the sensory organs, and they send signals to the spinal cord or brain. Motor neurons receive signals from the brain and spinal cord to control everything from muscle contractions to glandular output. Interneurons connect neurons to other neurons within the same region of the brain or spinal cord. When multiple neurons are functionally connected together, they form a neural circuit.

The term “GABAergic neurons” refers to neurons that are located when the hippocampus, thalamus, basal ganglia, hypothalamus, and brainstem. The balance between inhibitory neuronal transmission via GABA and excitatory neuronal transmission via glutamate is essential for proper cell membrane stability and neurologic function. Something is “GABAergic” if it pertains to or affects the neurotransmitter GABA.

The term “pain” refers to any unpleasant sensory experience, usually associated with a physical disorder. The physical disorder may or may not be apparent to a healthcare provider. Pain can be classified as one of two types: chronic and acute. An “acute pain” is a pain of short duration having a sudden onset. One type of acute pain, for example, is cutaneous pain felt on injury to the skin or other superficial tissues, such as caused by a cut or a burn. Cutaneous nociceptors terminate just below the skin, and due to the high concentration of nerve endings, produce a well-defined, localized pain of short duration. “Chronic pain” is a pain other than an acute pain. Chronic pain includes neuropathic pain, inflammatory pain, headache pain, somatic pain visceral pain, and referred pain.

The term “neuropathic pain” refers to an abnormal sensory input, resulting in discomfort, from the peripheral nervous system, central nervous system, or both. Symptoms of neuropathic pain can involve persistent, spontaneous pain, as well as allodynia (i.e., a painful response to a stimulus that normally is not painful), hyperalgesia (i.e., an accentuated response to a painful stimulus that usually causes only a mild discomfort, such as a pinprick), or hyperpathia (i.e., where a short discomfort becomes a prolonged severe pain). Neuropathic pain can be caused by, for example, a traumatic insult (e.g., a nerve compression injury such as a nerve crush, a nerve stretch, a nerve entrapment or an incomplete nerve transection), a spinal cord injury (e.g., a hemisection of the spinal cord), a limb amputation, a contusion, an inflammation (e.g., an inflammation of the spinal cord) or a surgical procedure. Neuropathic pain can also be caused by an ischemic event, an infectious agent, exposure to a toxic agent or a disease such as an inflammatory disorder, a neoplastic tumor, an acquired immune deficiency syndrome (AIDS), Lyme disease, a leprosy, a metabolic disease or a peripheral nerve disorder. Neuropathic pain also includes chronic pain, such as lower back pain, osteoarthritis and joint pain such as knee pain or carpal tunnel syndrome, myofascial pain and neuropathic pain.

Neuropathic pain can be related to a pain disorder, a term referring to a disease, disorder or condition associated with or caused by pain. Examples of pain disorders include arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

The term “pain disorder” refers to conditions or disorders which are secondary to disorders such as chronic pain and/or neuropathic pain (i.e., are influenced or caused by a disorder such as chronic pain and/or neuropathic pain).

The term “somatic pain” refers to pain that originates from ligaments, tendons, bones, blood vessels, and even nerves. It is detected with somatic nociceptors. The scarcity of pain receptors in these areas produces a dull, poorly localized pain of longer duration than cutaneous pain; examples include sprains and broken bones. Additional examples include the following: excessive muscle tension, repetitive motion disorders, muscle disorders, myalgia, infection, and drugs.

The term “neuralgia” refers to a pain that radiates along the course of one or more specific nerves usually without any demonstrable pathological change in the nerve structure. The various causes of neuralgia, including, chemical irritation, inflammation, trauma (including surgery), compression by nearby structures (e.g., from tumors), and infections. However, the cause is often unknown or unidentifiable. Neuralgia, includes, for example, a trigeminal neuralgia, a post-herpetic neuralgia, a postherpetic neuralgia, a glossopharyngeal neuralgia, a sciatica and an atypical facial pain.

The term “deafferentation” refers to a loss of the sensory input from a portion of the body and can be caused by interruption of either peripheral sensory fibers or nerves from the central nervous system. A deafferentation pain syndrome includes, for example, an injury to the brain or spinal cord, a post-stroke pain, a phantom pain, a paraplegia, a brachial plexus avulsion injuries, lumbar radiculopathies.

The term “light-sensitive protein” refers to a protein that is responsive to light. Membrane light-sensitive proteins can be activated with light, which leads to either a cation or anion exchange across the membrane that leads to either a hyperpolarization or depolarization of the membrane. Thus, depending on which protein is introduced and expressed, neural tissue can be either excited or depressed with light stimulation. Light-sensitive proteins can include membrane-bound light-sensitive ion channel or proton pump that leads to a hyperpolarization or depolarization of the cell as a function of light stimulation. Light-sensitive proteins include, for example, opsins such as rhodopsin, blue opsin, red opsin, halorhodopsin (NpHR), channelrhodopsin-2, enhanced halorhodopsin (eNpHR), archaerhodopsin-3 (Arch), Leptosphaeria maculans (Mac) and functional fragments or variants thereof. Light-sensitive opsins of the invention also include light-sensitive ion channels and ion pumps. A combination of two or more light-sensitive proteins can be used in the same method.

The term “optogenetics” refers to a biological technique to control the activity of neurons or other cell types with light. This is achieved by the expression of light-sensitive ion channels, pumps or enzymes specifically in the target cells. On the level of individual cells, light-activated enzymes and transcription factors allow precise control of biochemical signaling pathways. In systems neuroscience, the ability to control the activity of a genetically defined set of neurons has been used to understand their contribution to decision-making, learning, fear memory, mating, and addiction.

Optogenetics requires the introduction of opsin channel or pump proteins that respond rapidly to light in the plasma membranes of target neuronal cells allowing a temporarily accurate manipulation of the neuronal membrane potential while maintaining the resolution of the cell type through the use of specific mechanisms of targeting.

The term “opsins” refers to a group of proteins made light-sensitive via the chromophore retinal (or a variant) found in photoreceptor cells of the retina. Five classical groups of opsins are involved in vision, mediating the conversion of a photon of light into an electrochemical signal, the first step in the visual transduction cascade.

Multi-characteristic opsin (MCO) refers to an opsin with high photosensitivity with unique spectral and temporal characteristics to generate significant current in response to ambient light.

Channelrhodopsin-2 (ChR2) refers to an opsin that responds specifically to blue light. When ChR2 is inserted into neurons, blue light can be used to turn those neurons.

The term “halorhodopsin” refers to a light-gated ion pump, specific for chloride ions, found in archaea, known as halobacteria. It is a seven-transmembrane retinylidene protein from microbial rhodopsin family. It is similar in tertiary structure (but not primary sequence structure) to vertebrate rhodopsins, the pigments that sense light in the retina. Halorhodopsin contains the essential light-isomerizable vitamin A derivative all-trans-retinal. Halorhodopsin uses the energy of green/yellow light to move chloride ions into the cell, overcoming the membrane potential. Beside chlorides it transports other halides and nitrates into the cell. Potassium chloride uptake by cells helps to maintain osmotic balance during cell growth. By performing the same task, light-driven anion pumps can considerably reduce the use of metabolic energy. Halorhodopsin has been the subject of much study and its structure is accurately known. Its properties are similar to those of bacteriorhodopsin, and these two light-driven ion pumps transport cations and anions in opposite directions.

The term “GABAergic” refers to an agent that modifies the effects of GABA in the body or brain. Similarly, GABAergic neurons affect the neurotransmitter gamma-aminobutyric acid (GABA). For example, a synapse is GABAergic if it uses GABA as its neurotransmitter, and a GABAergic neuron produces GABA. Subclasses of GABAergic neurons include (a) Ca²⁺-binding protein parvalbumin (PVALB), (b) the neuropeptide somatostatin (SST), (c) vasoactive intestinal peptide (VIP) and (d) ionotropic 5-hyroxytryptamine 3a serotonin receptor (HTR3A).

The term “adeno-associated virus” or “AAV” refers to small viruses that infect humans and some other primate species. They are small (20 nm) replication-defective, nonenveloped viruses and have linear single-stranded DNA (ssDNA) genomes of approximately 4.8 kilobases (kb). Several features make AAV an attractive candidate for creating viral vectors for gene therapy and for the creation of isogenic human disease models. AAV-based vectors have emerged as the preferred vector system for neuro-logic gene therapy, with good safety record in clinical trials.

The term “nociception assay” refers to a technique to evaluate the ability of an animal (e.g., a mouse) to detect a noxious stimulus such as the feeling of pain, caused by stimulation of nociceptors. These assays measure the existence of pain through behaviors such as withdrawal, licking, immobility, and vocalization. The formalin assay is a common chemical assay of nociception which entails injection of a dilute solution of formalin into the surface of a rodent's hindpaw. Thereafter stereotypical behaviors such as flinching, licking, and biting of the affected hindpaw are monitored/scored.

The term “Von Frey assay” refers to a method that uses Von Frey hair or fibers (i.e., small pieces of nylon rod) to test a rodent's sensitivity to a mechanical stimulus. The von Frey test involves applying a punctate stimulus to a given region of the rodent's body, usually the plantar surface of the hind paw, and recording the stimulus intensity that evokes a withdrawal reflex.

The term “neurological disorder” broadly refers to a disorder of the nervous system. Neurological disorders can affect the brain as well as the nerves found throughout the human body and the spinal cord. Structural, biochemical, or electrical abnormalities in the brain, spinal cord, or other nerves can result in a range of symptoms. Neurological disorders include, for example, acute spinal cord injury, Alzheimer's disease, Amyotrophic Lateral Sclerosis (ALS), ataxia, Bell's Palsy, brain tumors, cerebral aneurysm, epilepsy and seizures, Guillain-Barré Syndrome, headache, head injury, hydrocephalus, meningitis, multiple sclerosis, muscular dystrophy, neurocutaneous syndromes, Parkinson's disease, stroke, headaches, encephalitis, and myasthenia gravis.

Neuroplasticity, also known as neural plasticity, or brain plasticity, is the ability of neural networks in the brain to change through growth and reorganization. These changes range from individual neuron pathways making new connections to systematic adjustments like cortical remapping. Examples of neuroplasticity include circuit and network changes that result from learning a new ability, environmental influences, practice, and psychological stress. Activity-dependent plasticity can have significant implications for healthy development, learning, memory, and recovery from brain damage.

The term “dendrite” refers to a branched protoplasmic extension of a nerve cell that propagates the electrochemical stimulation received from other neural cells to the cell body, or soma of the neuron from which the dendrite projects. Electrical stimulation is transmitted onto dendrites by upstream neurons (usually via their axons) via synapses which are located at various points throughout the dendritic tree. Dendrites appear to be capable of plastic changes during the adult life of animals, including invertebrates. Neuronal dendrites have various compartments known as functional units that are able to compute incoming stimuli. These functional units are involved in processing input and are composed of the subdomains of dendrites such as spines, branches, or groupings of branches. Therefore, plasticity that leads to changes in the dendrite structure will affect communication and processing in the cell. During development, dendrite morphology is shaped by intrinsic programs within the cell's genome and extrinsic factors such as signals from other cells. However in adult life, extrinsic signals become more influential and cause more significant changes in dendrite structure compared to intrinsic signals during development.

The term “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

The term “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

The term “under transcriptional control” is a term well understood in the art and indicates that transcription of a polynucleotide sequence, usually a DNA sequence, depends on its being operatively linked to an element that contributes to the initiation of, or promotes, transcription. “Operatively linked” means that the polynucleotides are arranged in a manner that allows them to function in a cell. In one aspect, this invention provides promoters operatively linked to the downstream sequences.

The term “encode” as it is applied to polynucleotides refers to a polynucleotide, which is said to “encode” a polypeptide if, in its native state or when manipulated by methods well known to those skilled in the art, it can be transcribed to produce the mRNA for the polypeptide and/or a fragment thereof. The antisense strand is the complement of such a nucleic acid, and the encoding sequence can be deduced therefrom.

The term “promoter” refers to a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, a MeCP2 promoter, a MeP418 promoter, an MeP426 promoter, a minimal MeCP2 promoter, a VMD2 promoter, an mRho promoter or an EFI promoter.

Additional promoters include, for example, EFIa, Ubc, human β-actin, CAG, TRE, Ac5, Polyhedrin, CaMKIIa, GalI, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6, and H1 promoters to abolish RNA polymerase III transcription and stimulate RNA polymerase II-dependent mRNA transcription). Synthetically-derived promoters may be used for ubiquitous or tissue-specific expression. Further, virus-derived promoters, some of which are noted above, may be useful in the methods disclosed herein, e.g., CMV, HIV, adenovirus, and AAV promoters. In embodiments, the promoter is used together with an enhancer to increase the transcription efficiency. Non-limiting examples of enhancers include an interstitial retinoid-binding protein (IRBP) enhancer, an RSV enhancer, or a CMV enhancer.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one that is placed in juxtaposition to a gene by means of genetic manipulation (i.e., molecular biological techniques) such that transcription of that gene is directed by the linked enhancer/promoter. Non-limiting examples of linked enhancer/promoters for use in the methods, compositions, and constructs provided herein include a PDE promoter plus IRBP enhancer or a CMV enhancer plus U1a promoter. It is understood in the art that enhancers can operate from a distance, and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

The term “homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW.

As used herein, amino acid modifications may be amino acid substitutions, amino acid deletions, or amino acid insertions. Amino acid substitutions may be conservative amino acid substitutions or non-conservative amino acid substitutions. A conservative replacement (also called a conservative mutation, a conservative substitution, or a conservative variation) is an amino acid replacement in a protein that changes a given amino acid to a different amino acid with similar biochemical properties (e.g., charge, hydrophobicity, or size). As used herein, “conservative variations” refer to the replacement of an amino acid residue by another, biologically similar residue. Examples of conservative variations include the substitution of one hydrophobic residue such as isoleucine, valine, leucine, or methionine for another; or the substitution of one charged or polar residue for another, such as the substitution of arginine for lysine, glutamic acid for aspartic acid, glutamine for asparagine, and the like. Other illustrative examples of conservative substitutions include the changes of: alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine; and the like.

The term “viral vector” refers to a recombinantly produced virus or viral particle that contains a polynucleotide to be delivered into a host cell, either in vivo, ex vivo, or in vitro. Examples of viral vectors include retroviral vectors, AAV vectors, lentiviral vectors, adenovirus vectors, alphavirus vectors, and the like. Alphavirus vectors, such as Semliki Forest virus-based vectors and Sindbis virus-based vectors, have also been developed for use in gene therapy and immunotherapy.

The term “recombinant expression system” or “recombinant vector” refers to a genetic construct or constructs for the expression of certain genetic material formed by recombination.

The term “gene delivery vehicle” refers to any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; bacteria; viruses, such as baculoviruses, adenoviruses, and retroviruses; bacteriophage, cosmid, plasmid, and fungal vectors; and other recombination vehicles typically used in the art which have been described for expression in a variety of eukaryotic and prokaryotic hosts, and may be used for gene therapy as well as for simple protein expression. Liposomes that also comprise, consist essentially of, or consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively, culturing conditions that can enhance the expression and/or promote the activity of the proteins disclosed herein are other non-limiting techniques.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating the transfer of genes to mammalian cells, as is known in the art and described herein.

The term “plasmid” refers to a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized before introduction into the host cell.

“Plasmids” used in genetic engineering can be referred to as “plasmid vectors.” Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene.

The term “adeno-associated virus” or “AAV” refers to a member of the class of viruses associated with this name and belonging to the genus Dependoparvovirus, family Parvoviridae. Adeno-associated virus is a single-stranded DNA virus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In embodiments, the AAV refers to the serotype AAV1, AAV2, AAV4, AAV5, AAV6, AAV7, AAV8, AAV9, AAV10, AAV11, AAV12, AAV13, AAVPHP.B, or AAVrh74.

The term “AAV vector” refers to a vector comprising, consisting essentially of, or consisting of one or more heterologous nucleic acid (HNA) sequences and one or more AAV inverted terminal repeat sequences (ITRs). Such AAV vectors can be replicated and packaged into infectious viral particles when present in a host cell that provides the functionality of rep and cap gene products; for example, by transfection of the host cell. In embodiments, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vector may also contain elements for manufacturing purposes, e.g., antibiotic resistance genes, etc., but these are not encapsidated and thus do not form part of the AAV particle.

The term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

An “AAV virion”, or “AAV viral particle”, or “AAV viral vector”, or “AAV vector particle” or “AAV particle” refers to a viral particle composed of at least one AAV capsid protein and an encapsidated polynucleotide AAV vector. Thus, the production of AAV vector particle necessarily includes the production of AAV vector, as such a vector is contained within an AAV vector particle.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well-known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

As used herein, the term “prevention” means all of the actions by which the occurrence of the disease is restrained or retarded.

The term “treating” or “treatment” refers to one or more of (1) inhibiting the disease (i.e., arresting further development of the pathology and/or symptomatology); and (2) ameliorating the disease (i.e., reversing the pathology and/or symptomatology) such as decreasing the severity of disease.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration.

The term “inductive charging” or “wireless charging” refers to a type of wireless power transfer. It uses electromagnetic induction to provide electricity to portable devices. Inductive charging is also used in vehicles, power tools, electric toothbrushes, and medical devices. The portable equipment can be placed near a charging station or inductive pad without needing to be precisely aligned or make electrical contact with a dock or plug. Inductive charging is named so because it transfers energy through inductive coupling. First, alternating current passes through an induction coil in the charging station or pad. The moving electric charge creates a magnetic field, which fluctuates in strength because the electric current's amplitude is fluctuating. This changing magnetic field creates an alternating electric current in the portable device's induction coil, which in turn passes through a rectifier to convert it to direct current. Finally, the direct current charges a battery or provides operating power.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

While pain syndromes arise from peripheral input and are potentially amenable to spinal cord manipulations, other conditions (i.e. central pain syndromes) arise from changes in supraspinal functions. Embodiments include methods of targeting higher-level neurons (e.g., cells of the spinal cord, DRG and ACC) in the central nervous system (CNS) to reduce pain.

The role of specific structures of the CNS in pain is complex as the study of this anatomy and its function in pain processing is confounded by its essential role in life as well as the interconnectedness of various substructures for the formation of a functional CNS. Despite this, multiple studies have established the role of major structures in pain such as the DRG, thalamus (i.e., for routing pain information), the insula, and the anterior cingulate cortex (ACC). Studies in suggest that neurons of the DRG and ACC are involved in pain perception (among many other functions of the DRG and ACC), specifically the affective-aversive aspects of the pain experience.

The ACC receives input from multiple brain regions, including the thalamus, hippocampus amygdala and other areas in the cortex. It is apparent from electrophysiological analysis that neurons in the ACC respond to noxious stimuli and are active when pain is anticipated. Moreover, activation of the ACC can reduce pain behavior and activity in the dorsal horn. These findings have been confirmed with neuroimaging studies that show that the ACC and other cortical structures are activated by noxious stimuli, including psychosocial pain. Lesions to the ACC have been associated with reductions in pain responses in animal models. Persistent chronic pain has also been shown to manifest in plasticity changes in the ACC. Alterations in the balance between excitation to inhibition (i.e., the E:I ratio) by long-term potentiation of pyramidal neurons, decreased activity of interneurons, or loss of inhibitory modulation can lead to persistent pain. As the ACC is involved in multiple other functions such as social behavior, it has been proposed that modulation (such as through electrical stimulation) rather than lesioning could be viable as a pain control option.

Abnormalities of the cortical excitation to inhibition (E/i) balance can also play a role in disorders such as autism, including such comorbidities such as reduced pain sensitivity. Previous research has determined that under pain conditions, the E/I balance of the ACC shifts towards inhibition. Thus, optogenetic enhancement of inhibitory GABAergic neuron activity can lower the E/I ratio as a strategy for pain treatment. In previous studies, it was demonstrated that optogenetic manipulation of central brain structures related to pain (e.g., ACC) alters pain sensation measured using reflexive behavioral assays. The proposed mechanism for these results in the ACC is the modulation of the cellular E/I balance.

Previous studies have demonstrated that optogenetic stimulation of inhibitory neurons in the ACC leads to decreased electrical activity in the ACC and significant reductions in pain responses due to skin irritation and noxious stimulation. Importantly, modulation of ACC function by electrical stimulation is intrinsically limited as such stimulation can lead to altered excitability in both excitatory and inhibitory interneurons that can lead to an exacerbation of the mental state generated by the pain stimulus. An alternate approach as described herein is the use of optogenetics.

The challenges with electrode-based brain stimulation in alleviating pain can be attributed to the limitations of electrical stimulation methods as they are inherently nonspecific. No particular cell type can be stimulated without affecting the neighboring cells. Furthermore, pharmaceutical drugs are similarly nonspecific. For example, bicuculine has major disadvantages: it is a competitive inhibitor (i.e., its efficacy will depend on the local GABAergic tone of the area) and it is unstable. In order to address these shortcomings, Applicants utilize the optogenetic methods described herein in which GABAergic neurons are targeted specifically via promoter-specific expression. The use of optogenetics with precise fiber-optic stimulation allows localized modulation of with controlled stimulation frequencies. The results demonstrate that continuous optogenetic stimulation has the strongest effect on inflammatory pain in the latter stages of the formalin pain response. Moreover, optogenetic modulation of chronic pain only has a strong effect on behavior in a biased (light-dark) conditional placement preference when the modulation was applied in real-time. The bright light may have itself caused pain which would minimize the strength of post-conditioning preferences.

Applicants present herein a novel and effective approach for pain modulation by focusing on the modulatory and communication pathways that connect the sites of injury/pain with the cortical perception structures. In embodiments, the spine (e.g., the neurons of the spinal cord, ACC and/or DRG) is targeted with a cell-specific treatment (e.g., via promoter-driven opsin-gene expression).

Embodiments of the invention include compositions and methods for the specific delivery of light-sensitive proteins to sensory neurons in the ACC to optically silence pain-associated neurotransmission, resulting in reduced sensation of chronic pain. This approach provides control of activities in specifically targeted families of neurons in a spatially and temporally specific manner using light.

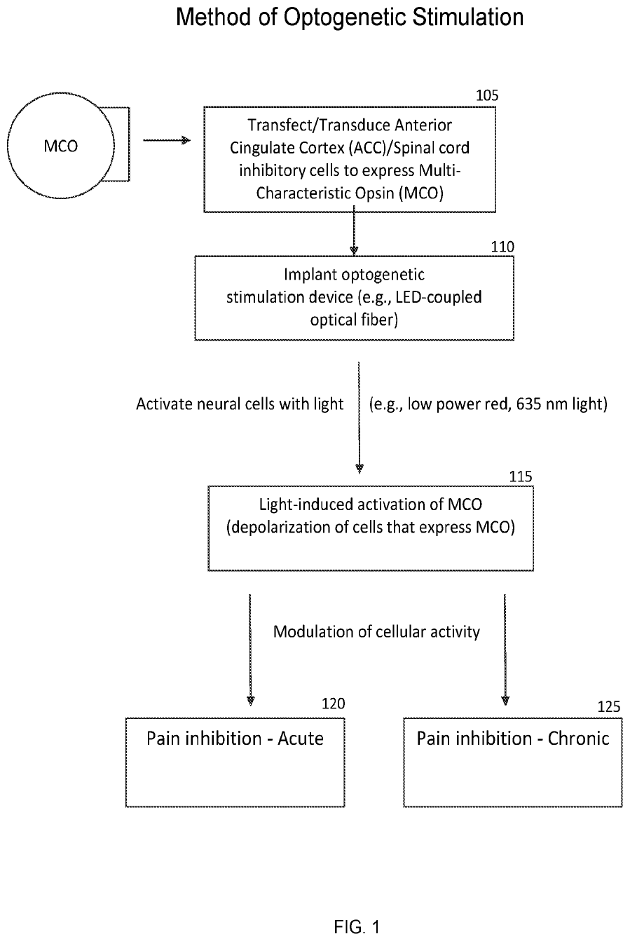

FIG. 1 is a flowchart that summarizes the steps of a method of optogenetic stimulation described herein. In the first step (105), neural cells are transfected with a virus to express multi-characteristic opsin (MCO). In some aspects, the neural cells are cells of the spinal cord, cells of the dorsal root ganglia (DRG) or cells of the anterior cingulate cortex (ACC).

The method can also include the implantation of a wireless optogenetic stimulation device (110) such as an optical fiber or similar light source. The implantable device can be made of glass, polymer, PMMA, silicone or PDMS; and coated with a biocompatible material to minimize neuro-inflammatory response without compromising the light guidance. While transdermal transmission of light can be used, an optical fiber avoids the limitations of external light sources (e.g., heat transmission). In some aspects, the light source is activated via a wireless signal. In some aspects, the light source is charged wirelessly (e.g., via inductive charging) thus avoiding the requirement of an internal or linked power source.

The light device can activate the transfected neural cells using visible light. Light-induced activation of multi-characteristic opsin (MCO) results in depolarization of only those cells that express MCO (115). Thereafter, the subject (e.g., mouse) can be studied by pain scoring for acute (120) and chronic (125) pain.

The methods described herein can use optogenetics to enable neuronal stimulation in a highly selective manner with millisecond-level temporal precision. This opsin-based approach can be applied to pain modulation as well as conditions such as visual impairment, drug addiction, psychological disorders, or movement disorders. Through the use of opsins, the sensitization of cells to specific wavelengths of light creates a means by which cells related to the essential aspects of pain syndromes (e.g., from nociception to allodynia) can be modulated.

Embodiments also include a wireless (e.g., BLUETOOTH™) optogenetic pain modulator device which can regulate the frequency and intensity of optogenetic stimulation. The sensitivity of the optogenetic actuator, multi-characteristic opsin (MCO), minimizes the power requirements for pain modulation by activation with low intensity light. The sensitivity of MCO to wavelengths in the red spectrum of visible light, with deeper tissue penetration, allows in-depth activation with a more shallowly implanted light. As described in the below examples, stimulation of inhibitory neurons in the spinal cord, DRG, or ACC expressing MCO led to reduction of reflexive acute pain responses and changes in conditioned placement preference in a mouse model of chronic pain. Additionally, measurements demonstrate inhibition of pain responses is dependent on the DRG/ACC optogenetic stimulation schedule. The results described herein support modulation of the inhibitory pathways within the DRG/ACC as a viable alternative for inhibiting chronic neuropathic pain.

The methods described herein can be used to treat numerous disorders related to chronic pain and central sensitization including, for example, fibromyalgia, rheumatoid arthritis, osteoarthritis, chronic arthropathy, spinal nerve compression syndromes associated with neoplasia and/or disc herniation, chronic back pain, chronic joint pain of any etiology associated with inflammation and/or structural joint abnormalities, post-herpetic neuralgia, trigeminal neuralgia, chronic metabolic neuropathy associated with chronic pain, migraine, inflammatory pain, post-surgical pain syndromes including phantom limb pain, post-traumatic stress disorder (PTSD), irritable bowel syndrome, autonomic neuropathies, arachnoiditis, chronic regional pain syndrome, vulvodynia, and chronic pain syndrome associated with activation of central sensitization pathways.

The methods can be used as a preventive measure (i.e., to avoid an ailment) at one time or multiple times. Alternatively, methods can be employed over a series at any time from diagnosis onwards. The method can be a sole treatment or used in conjunction with other drugs or therapies useful in treating the condition in question.

### Administration

Channelrhodopsin-2 (ChR2) is a non-selective cation channel that is used for the depolarization of neurons upon activation with light. Selective activation (ChR2) and silencing of neurons by ms-pulsed light pulses have been demonstrated in cell culture, brain slices, and in animals. Currently, optogenetic approaches for neuronal modulation utilizing ChR2 or Halorhodopsin (NpHR, a chloride channel) require moderate light intensity, which raises the possibility of phototoxic damage to cells in the area. Multi-Characteristic Opsin (MCO) is a novel optogenetic molecule having red-light sensitivity, which is valuable for the sensitization of deep tissues as light in these wavelengths propagates through tissues without attenuation or cytotoxicity. MCO combines this wavelength specificity with a high sensitivity and allows effective stimulation with very low-power light.

Embodiments also include vectors with a nucleotide sequence encoding a light-sensitive opsin protein or any variant thereof described herein (e.g., a rhodopsin). In aspects, the opsin is identified as SEQ ID NO:1. Vectors that can be administered according to the invention also include vectors that include a nucleotide sequence encoding an DNA, RNA (e.g., an mRNA), siRNA that when transcribed from the vector polynucleotides results in the expression of light-sensitive proteins in the plasma membranes of the target animal cells. Vectors that can be used include, for example, lentiviral vectors, HSV, adenoviral, and adeno-associated virus (AAV). Lentiviruses include HIV-1, HIV-2, VIS, VIF and VAIE. Lentiviruses can be pseudotyped with the envelope proteins of other viruses, including VSV, rabies, Mo-MLV, baculovirus, and Ebola viruses. Such vectors can be prepared using conventional methods in the art.

In some embodiments, the vector is a recombinant AAV vector. AAV vectors are relatively small-sized DNA viruses that can be integrated, in a stable and site-specific manner, into the genome of the cells they infect. They are capable of infecting a broad spectrum of cells without inducing any effect on growth, morphology, or cell differentiation and do not appear to be involved in human pathologies. The AAV genome has been cloned, sequenced, and characterized. It covers approximately 47.00 bases and contains an inverted terminal repeat (ITR) region of approximately 145 bases at each end, which serves as a source of replication for the virus. The rest of the genome is divided into two essential regions that carry the functions of encapsidation: the left part of the genome, which contains the rep gene involved in viral replication and the expression of viral genes, and the right part of the genome, which contains the Cap gene that encodes the capsid proteins of the virus. AAV vectors can be prepared using standard methods in the art. The AAV recombinants that are produced are then purified by standard techniques.

In some embodiments, the vector for use in the methods of the invention is encapsulated in a virus particle (e.g., VAA virus particle, including, for example, AAV 1, AAV 2, AAV 3, AAV 4, AAV 5, AAV 6, AAV 7, AAV 8, AAV 9, AAV 10, AAV 11, AAV 12, AAV 13, AAV 14, AAV 15 and AAV 16). Accordingly, the invention includes a recombinant virus particle (recombinant because it contains a recombinant polynucleotide) comprising any of the vectors described herein. The methods for producing such particles are known in the art.

In embodiments, the promoter used to drive expression of MCO targets inhibitory neurons. Suitable promoters for such inhibitory GABAergic neurons include, for example, GAD65/67, VGAT, SLC32A1, GAD1 and GAD2. In some aspects, an enhancer element is included (e.g., DLX 1, DLX 2, DLX 5 or DLX 6). Suitable promoters for DRG cells include, for example, SV2 and MAP1B. Suitable promoters for satellite glial cells include Bestrophin 1, Glial Fibrillary Acidic Protein, Bcan, Fdps, MIc1, Gja1, Ednrb, SIc1a3, PIp1, and Fabp7.

Certain embodiments of the invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The therapeutic agents in the pharmaceutical compositions can be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount may vary depending on the condition to be treated, the severity and course of the condition, the mode of administration, whether the agent is administered for preventive or therapeutic purposes, the bioavailability of the particular agent(s), the ability of the therapeutic small molecule to elicit a desired response in the individual, previous therapy, the age, weight and sex of the patient, the patient's clinical history and response to the agent, the type of the therapeutic small molecule used, discretion of the attending physician, etc. A therapeutically effective amount is also one in which any toxic or detrimental effects is outweighed by the therapeutically beneficial effects. A “prophylactically effective amount” refers to an amount effective, at dosages and for periods of time necessary, to achieve the desired prophylactic result.

In aspects of this embodiment, a method of the invention reduces the incidence of pain by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a method disclosed herein reduces the incidence of pain from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

Aspects of the present specification disclose, in part, treating an individual who is susceptible to pain or suffering from pain (acute or chronic). As used herein, the term “treating,” refers to reducing or eliminating the pain; or lowering or depleting the incidence of pain. For example, the term “treating” can mean reducing pain by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. Those of skill in the art will know the appropriate symptoms or indicators associated with a specific type of ailment and will know how to determine if an individual is a candidate for treatment as disclosed herein.

In aspects of this embodiment, a method disclosed herein reduces pain, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a method disclosed herein reduces pain by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a method herein reduces pain by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

### Methods

Production of GAD67-MCO2-mCherry and Packaging into Viral Vector

pAAV-GAD67-MCO2-mCherry plasmid was designed by Nanoscope Technologies LLC. The plasmid was packaged into an AAV5 (hereafter referred to as pMCO2) using triple transduction method.

### Mouse Models

C57BL/6J (wild type) male and female mice were obtained from Jackson Laboratory. The experimental mice were maintained on a 12:12 light cycle and in strict compliance with IACUC guidelines for the use of animals in research. The mice were housed and experimented humanely.

Fiber Optic Stimulation Implant and ACC Transduction with AAV-MCO2 in Wt Mice

Aseptic technique was used for all surgical procedures and surgical tools were sterilized in an autoclave. The wild-type (wt) mice were anesthetized with 2-3.5% isoflurane and the fur over the area of interest was removed chemically. A midline incision was made, and the skin was removed in the ACC area. A burr hole was made over the ACC (0.7 mm anterior to bregma, 0.4 mm lateral from the midline, and at a depth of 1.8 mm from the skull surface), and a 1.5 mm long implant was installed and secured to the skull with cyanoacrylate and dental cement. AAV-MCO2 was injected into the ACC at this point with a syringe (dose-dependent). The mouse was maintained for two weeks with the implant to allow proper expression and then used for experimentation. The novel BLUETOOTH™-controlled, back-mounted, optogenetic stimulation device activated the ACC via this permanently implanted cannula.

DRG Transduction with AAV-Carried MCO2 in Wt Mice

Aseptic technique was used for all surgical procedures and surgical tools were sterilized in an autoclave. The wild-type (wt) mice were anesthetized with 2-3.5% isoflurane and the fur over the area of interest was removed chemically. AAV-carried MCO2 was delivered intrathecally.

Spinal Transfection with Laser Delivered pMCO2 in Wt Mice

Aseptic technique was used for all surgical procedures and surgical tools were sterilized in an autoclave. The wild-type (wt) mice were anesthetized with 2-3.5% isoflurane and the fur over the area of interest was removed chemically. Syringes were aseptically filled, and anesthetized animals were given a single intrathecal injection of gold nanorods (GNRs) mixed with MCO plasmids. 10 μL of the transfection solution at the appropriate dose concentration was drawn into a 20 μL Hamilton syringe. Convection-enhanced delivery infusions were carried out for ~10 min at a flow rate of ~1 μl/min through the spine. After completion of the procedure, the solution was left in place to permeate the spine for 2 hours. The spine was irradiated with 1080 nm light.

### Formalin Assay

Mice were given a 20 microliter injection of 1% Formalin and placed in a holding chamber. At five-minute intervals, for one minute each, mice were observed for paw lifting and paw licking behavior. The total time in seconds for each minute of observation wherein this behavior was observed was recorded. This was continued for 45 minutes. Pain scoring was performed according to the following formula:

$$( ( 2 * Paw Licking time ) + ( 1 * Paw Lifting time ) ) / 60$$

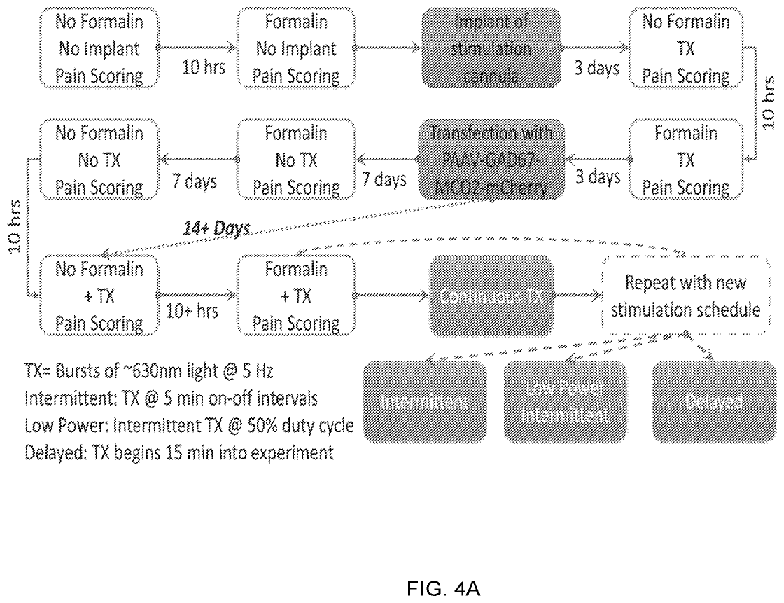

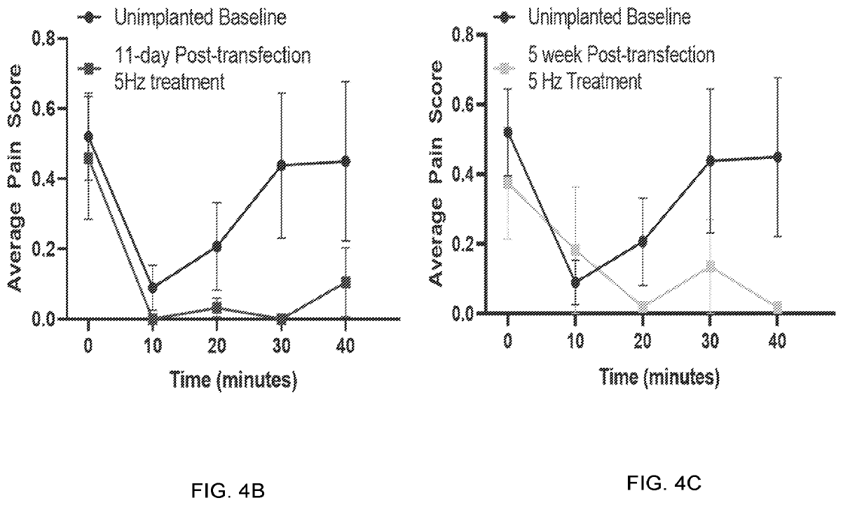

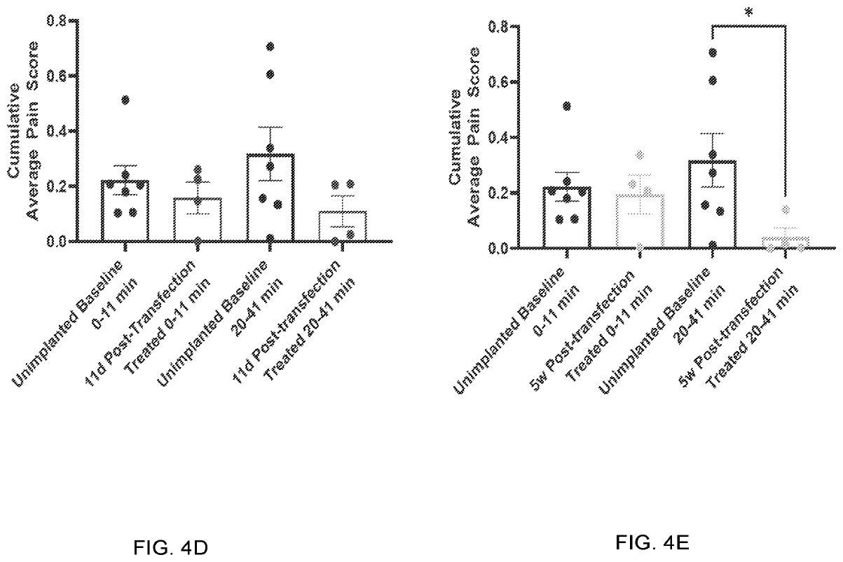

This was repeated in implanted wt mice with and without stimulation (2 or 5 Hz at ~630 nm on varying schedules) to determine any statistically measurable difference in activity. The experimental design is outlined in FIG. 4A.

### Chronic Pain Induced by Sciatic Nerve Ligation

Mice were anesthetized with isoflurane or intraperitoneal injection of 4 ml/kg of a mixture of ketamine (17 mg/ml) and xylazine (2.5 mg/ml) in sterile Phosphate Buffered Saline (PBS). Fur was removed chemically from one hind leg. The surgical area was cleaned with 70% Ethanol. The sciatic nerve was exposed through a muscle-sparing incision along the sciatic vein between the semitendinosus and the biceps muscles. The two muscles were gently spread to expose 1.5 cm of sciatic nerve. A sterile glass hook was used to lift the main branch of the sciatic nerve. The sciatic nerve was hydrated with sterile PBS. Using a 4-0 silk suture the main branch of the sciatic nerve was tightly ligated. The muscles were then sutured using a 4-0 suture. The overlaying skin was closed using staples. The mouse was allowed to recover on a warm pad. By the day following the ligation, and for several months thereafter, the mice develop guarding behavior of the ipsilateral hind paw to mechanical stimulation. This is often used as a model of neuropathy-induced pain. This mechanical allodynia is accompanied with thermal hyperalgesia lasting approximately three weeks.

### Conditioned Placement Preference Assay

Pre-conditioning phase: Sciatic nerve ligation was performed on all mice to be used for testing. During this phase (day 1 to 3), the guillotine doors were removed to allow free access to the entire apparatus. The experimental mouse was placed in the middle chamber. Each experimental animal's unfettered movement through the chambers was recorded for 15 minutes. The time spent in the two side chambers on the third day is the baseline preference. Any mice that entered less than four times to either of the side chambers were removed from consideration.

Conditioning phase: During conditioning (day 4 to 9), the mice were confined within the treatment (lit) chamber or the unlit (no treatment) chamber for 45 minutes. 10 hours later each mouse was confined within the opposite chamber from their morning association session for 45 minutes. While in the treatment-associated chamber, the mouse was given optogenetic stimulation at a rate of 5 Hz. When in the non-treatment paired chamber, the mounted BLUETOOTH™ stimulation device was placed on the mouse but not active.

Testing phase: During testing, the mouse was placed in the middle passage and allowed free access to the entire apparatus. During their exploration, their activity was recorded for 15 min. These post-conditioning tests were carried out 3, 6, and 10 days after conditioning. During live testing, the mouse was placed in the middle passage as normal but the optogenetic stimulation was turned on whenever the mouse entered the lit chamber and turned off when it was in the middle passage or the unlit chamber.

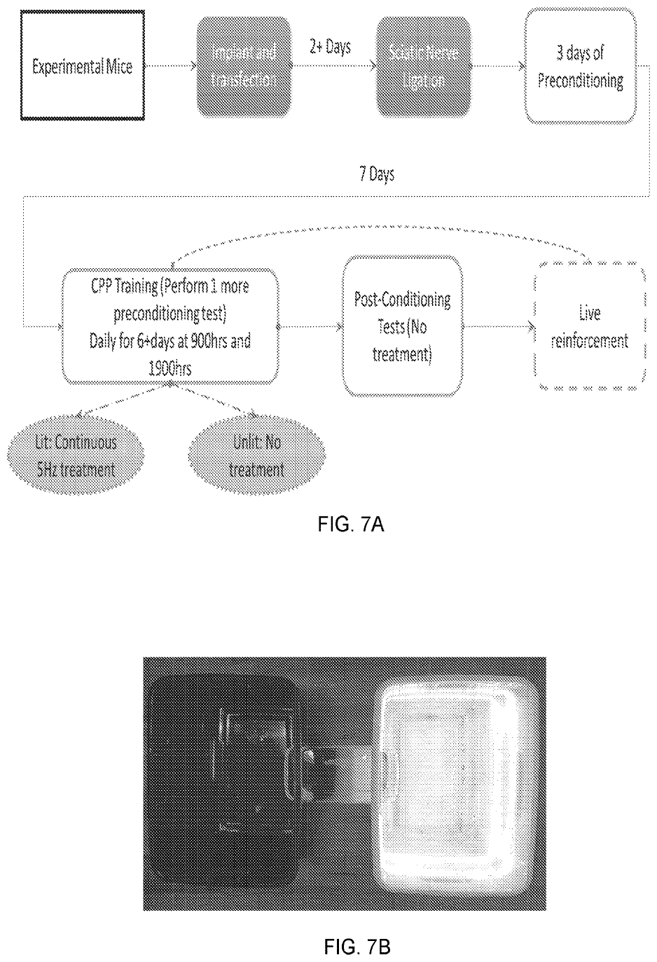

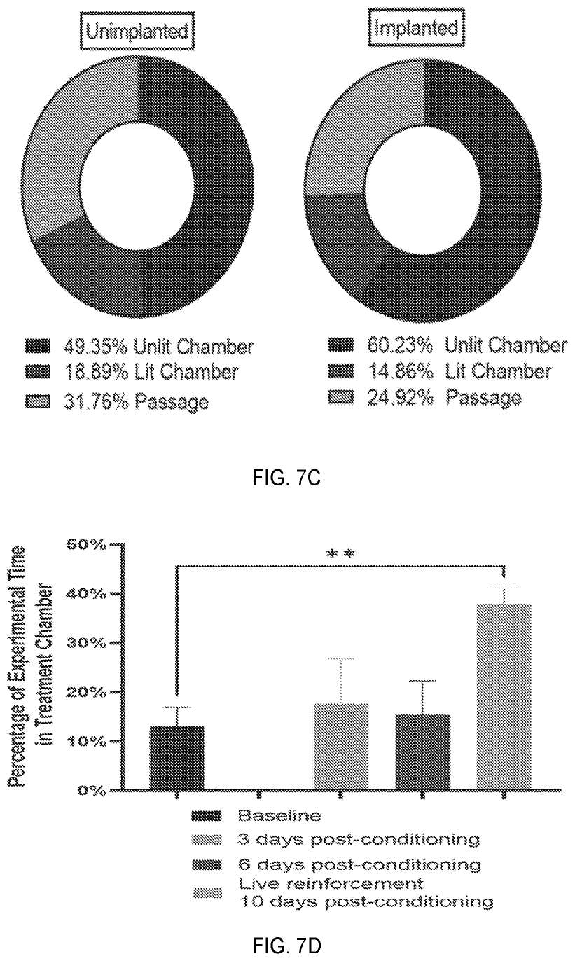

The percentage of time spent in either chamber (“chamber time”) of the entire 15-minute testing session at each time point was calculated. The overall experimental outline and CPP apparatus are illustrated in FIG. 7B.

### Von Frey Assay

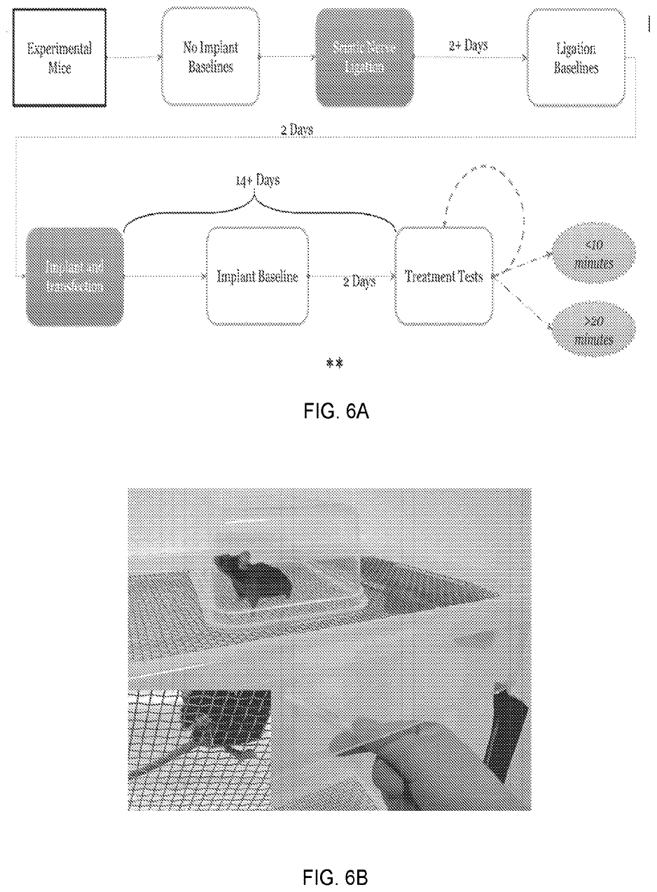

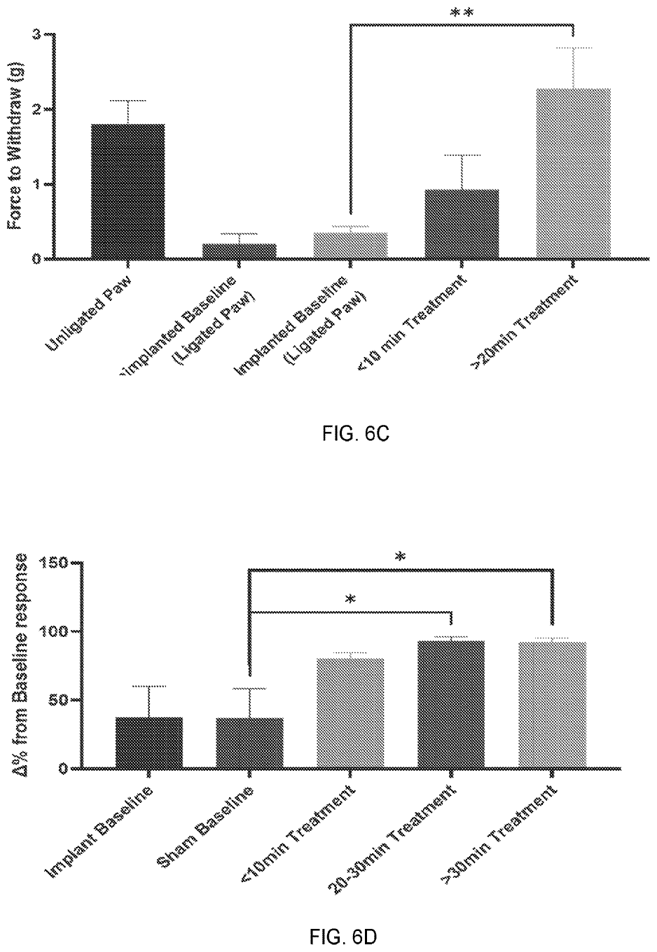

Baseline mechanical thresholds for withdrawal response were established for each experimental mouse via an increasing or decreasing presentation method. After an acclimatization period of at least 30 minutes, the testing phase began and optogenetic stimulation was initiated if applicable for the test. Starting at 0.6 g the hindpaw was stimulated with a von Frey filament (pressing the tip to the plantar surface until it bent). This presentation was repeated five times and the presence or absence of a response was noted. Two minutes of time was left between each presentation. If at least three of the five results were positive then a lower filament was used, if less than three of the five presentations were positive for a withdrawal response then the next higher filament was used. This was continued until a minimum threshold for at least three out of five results being positive was established. After sciatic nerve constriction (see above), the mouse hindpaw was tested in the same manner. For timed treatment, the methodology was adjusted to the standard “up-down” method in which a test was immediately followed (with approximately a one-minute gap) with the next higher or lower filament depending on if the presentation was negative or positive (respectively) for a withdrawal response. This continued until at least 3 presentations were positive for a particular filament. This was necessary as the normal 2 minutes would not allow the test to be completed in a timely manner. The overall experimental process and Von Frey assay setup are shown in FIG. 6A and FIG. 6B respectively.

### Immunostaining of Tissues for Immunogenicity and Viability Assay

Mice were sacrificed and their brains extracted and placed in PFA for 8 hours. The brains were then moved to 30% sucrose (w/v) and until cryoprotected. The brains were then sectioned and collected sections were stained with either anti-IBA1, anti-CD45, Anti-Gad65 (1:100), or anti-Caspase (1:250) primary antibodies as well as anti-MCherry (1:250 or 1:500). Secondary antibodies (1:500) were added after overnight incubation with the primary antibodies and finally each slide was stained with DAPI, and a coverslip was placed over the samples. Each was then imaged via confocal microscopy.

### Statistical Analysis

The collected data from the experimental rodents was analyzed using GraphPad Prism 9.3 (for Windows, GraphPad Software, San Diego, California USA). Statistical analysis was performed via a parametric T-test.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

Wireless Optogenetic Stimulation Device Allows Pain Modulation in Freely Moving Animals with a Minimally Invasive Implant.

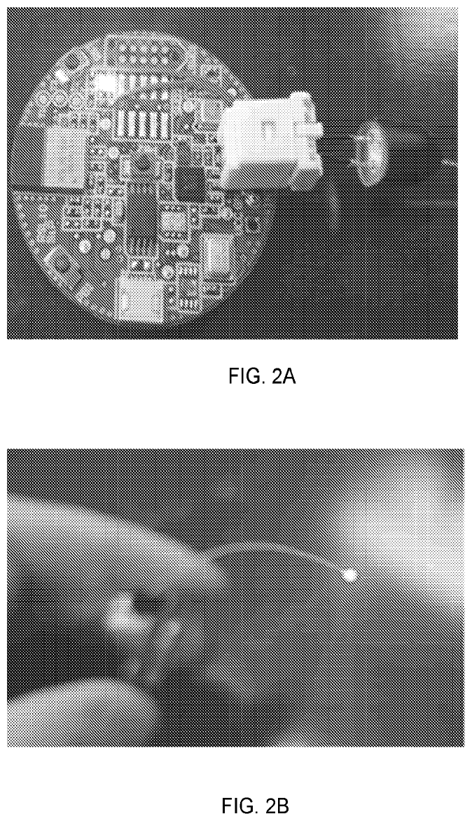

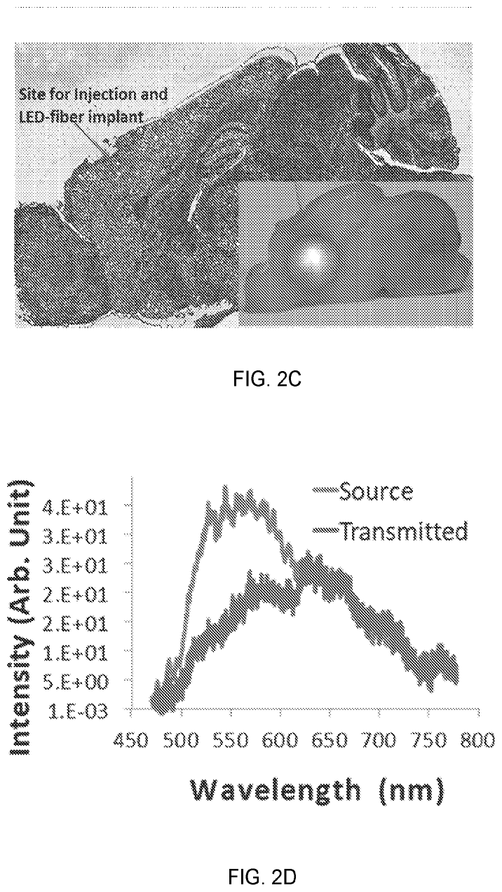

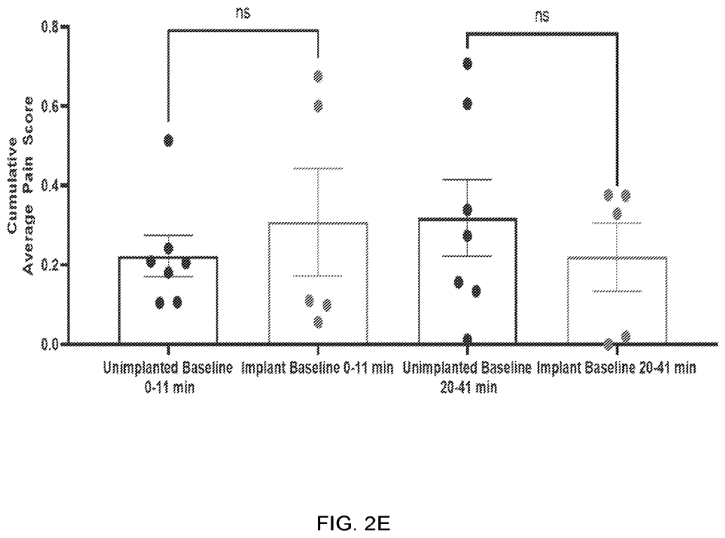

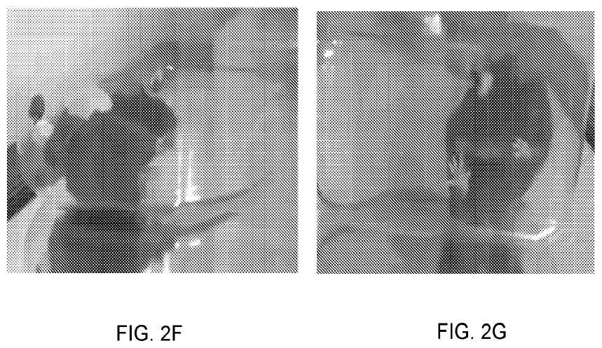

Optogenetic stimulation apparatus allows freely mobile optogenetic modulation of pain with a minimally invasive implant that does not significantly affect pain response. FIG. 2A-FIG. 2B shows that a minimally invasive wireless optogenetic stimulation device. The device allows freely mobile optogenetic modulation of pain that does not significantly affect pain response. FIG. 2A is an image of the BLUETOOTH™-enabled optogenetic stimulation device for wireless control of optogenetic modulation of pain. FIG. 2B is an image of an optical fiber that emits low-power red (i.e., 635 nm) light. The optical fiber emits low-power red light while being minimally invasive. This is due to the propagation of red light through brain tissue. FIG. 2C is an image of a sagittal section of a mouse brain showing minimal invasion by optical fiber (marked by an arrow) on the dorsal side of the ACC. FIG. 2D shows the normalized transmission spectrum of visible light through a 1 mm thick brain slice. This is a graphical depiction of the propagation of red light through brain tissues. Minimal absorption is observed above 630 nm. FIG. 2E demonstrates that the presence of the fiber implant did not significantly affect acute pain responses in formalin assay as measured by quantifiable (ns: not significant) behaviors such as licking (FIG. 2F) and paw lifting (FIG. 2G).

### Example 2

### Co-Localization of MCO2-mCherry in the ACC GABAergic Neurons

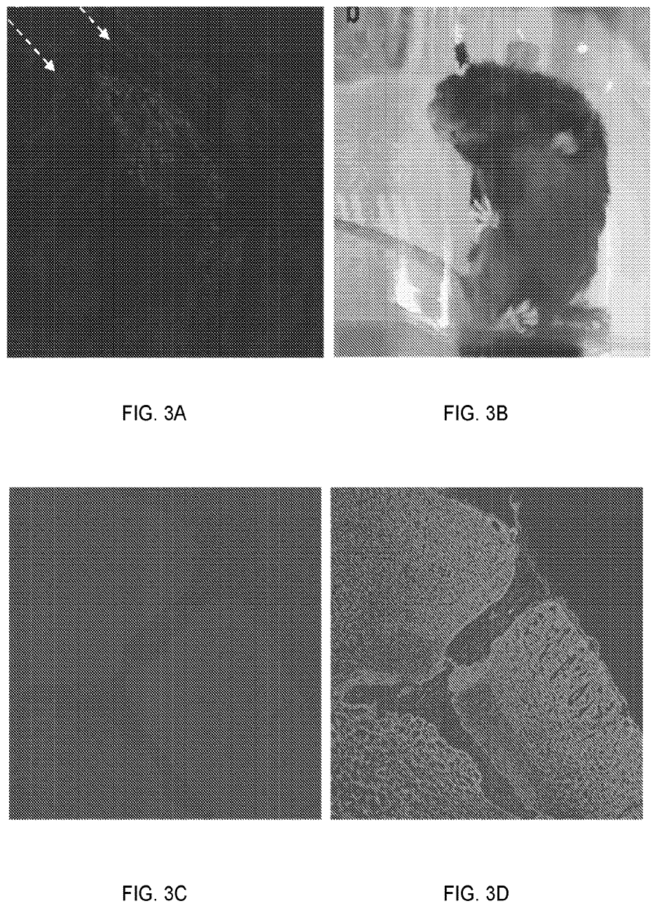

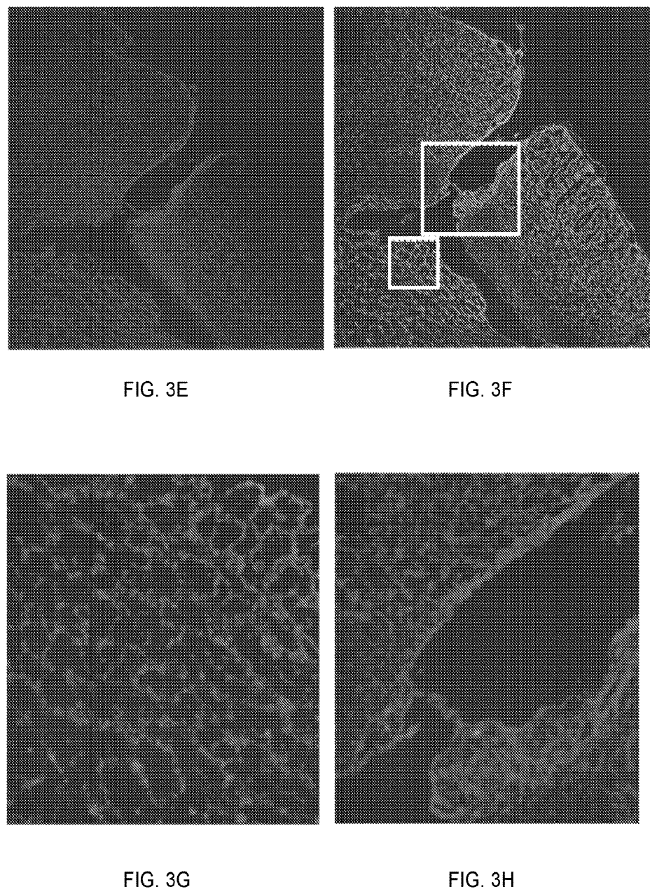

To examine the expression of MCO2 in anterior cingulate cortex (ACC), the sections of pMCO2 transduced brain were immunostained with cell-specific antibodies, along with the GFP antibody. Analysis of sectioned brain tissues showed transduction of the ACC colocalized with GAD65-labelled cells. Intrinsic mCherry fluorescence imaging of GAD67 promoter-driven MCO expression in the ACC was conducted two weeks after pMCO2 injection. Optical fiber outline is indicated by arrows. FIG. 3A shows GAD65 and mCherry positive labeled cells in the ACC. FIG. 3B is an image of a wireless fiber-coupled red LED implanted in MCO-transfected mouse.

FIG. 3C-FIG. 3E are confocal immunostained images of mouse brain slices. FIG. 3C is a DAPI (nuclear stain). FIG. 3D is a GAD65-marker for GABAergic neurons and FIG. 3E an Multi-Characteristic Opsin (MCO) Reporter-mCherry. FIG. 3F is an overlay of GAD65 & mCherry. Analysis of sectioned brain tissues showed transduction of the ACC colocalized with GAD65-labelled cells. Zoomed areas are indicated by the marked rectangles are shown in FIG. 3G and FIG. 3H. The images show high magnification co-expression image of the ACC and colocalization of GAD65 and mCherry.

### Example 3

Acute Inflammatory Pain Responses are Diminished by Optogenetic Stimulation with MCO-Transduction of ACC GABAergic Neurons

The next example involves testing mice for responses to pain with optogenetic stimulation. FIG. 4A is a flowchart of an experimental plan for assessing effect of optogenetic modulation on acute pain model (i.e., formalin injected to hind paw). Baseline formalin induced pain scores at 10-minute intervals were compared with that during continuous 5 Hz optogenetic stimulation (5 ms pulses at 630 nm). FIG. 4B shows the results 11 days after early transduction; FIG. 4C shows the results five weeks after optimal transduction. The charts show average of the cumulative pain scores (measured in 5-minute intervals) in the early (0-11 minutes) non-inflammatory and late (20-41 minutes) inflammatory phases of pain. FIG. 4D shows the results 11 days early post-transduction; FIG. 4E shows the results 5 weeks post optimal transduction. Av.±SEM. N=7 for baseline and 4 for MCO-transduced group (mice with incorrectly placed fiber were excluded). * p<0.05.

### Example 4

### Optogenetic Stimulation of ACC GABAergic Neurons Reduced Acute Pain Behavior

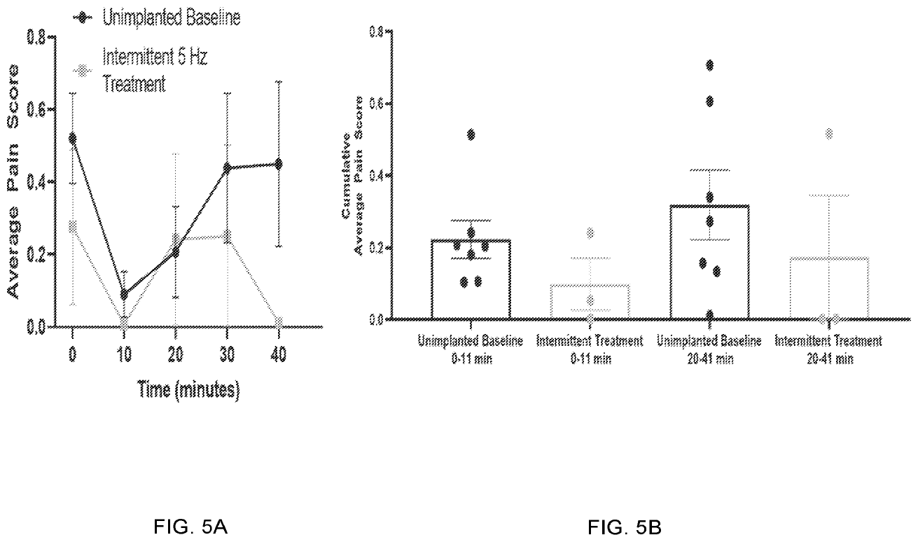

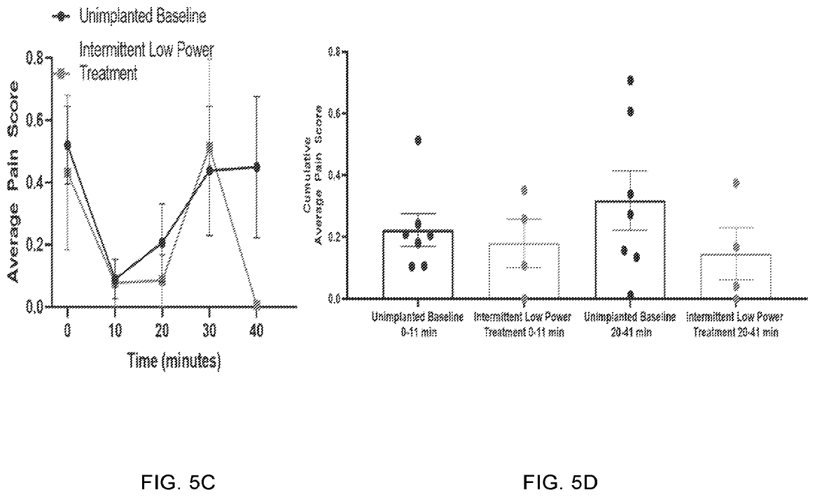

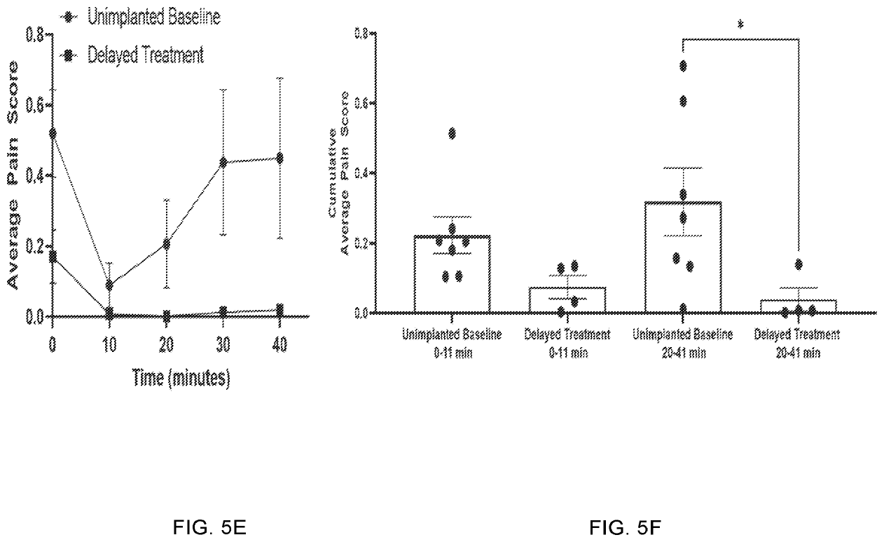

Untreated mice also showed greater pain scores overall as compared to mice with intermittent treatment. During the formalin assay, experimental mice were given 5 Hz optogenetic stimulation on a 5-minute on-off schedule (FIG. 5A-FIG. 5D). The effect of this treatment schedule on the late phase of pain is reduced when compared to the 5-week post-transduction late phase pain response with constant treatment (from an 87.4292% decrease to a 45.5202% decrease).

The results of formalin studies are shown in FIG. 5A-FIG. 5F. Baseline formalin-induced pain scores at 10-minute intervals compared with: (FIG. 5A) 5 Hz optogenetic stimulation (5 ms pulses at 630 nm) for 5-minute on-off intervals; (FIG. 5B) 2 Hz optogenetic stimulation for 5-minute on-off intervals; and (FIG. 5C) with 5 Hz optogenetic stimulation starting 15 minutes into the experimental session. The average of the cumulative pain scores (measured in 5-minute intervals) in the early (0-11 minutes) non-inflammatory and late (20-41 minutes) inflammatory phases of pain: (FIG. 5D) with intermittent 5 Hz optogenetic stimulation; (FIG. 5E) intermittent 2 Hz optogenetic stimulation; and (FIG. 5F) with delayed 5 Hz optogenetic stimulation. Av.±SEM. N=7 for baseline and 4 for the MCO-transduced group (mice with incorrectly placed fiber were excluded), * p<0.05.

### Example 5

### Mechanical Hyperalgesia and Allodynia are Reduced in Chronic Pain Model by MCO-Based Optogenetic Stimulation of the ACC GABAergic Neurons.

Mechanical Hyperalgesia and Allodynia are reduced relative to baseline thresholds with MCO-based optogenetic stimulation of the ACC. FIG. 6A is a flow chart of an experimental plan for mechanical hypersensitivity chronic pain experimentation. FIG. 6B is an image of a manual Von Frey apparatus showing the presentation of a Von Frey filament to the hindpaw of a baseline mouse. The inset shows the Von Frey filament making contact with the mouse paw. FIG. 6C shows that the minimum force needed to elicit hindpaw withdrawal response to mechanical stimulation increases with increased duration of exposure. FIG. 6D shows the percent change in withdrawal force relative to the mouse's untreated baseline threshold. N=4. Avg±SEM. *=p<0.05, ** p≤0.01.

### Example 6

### Conditioned Placement Preference Responses to Implantation and MCO-Based Optogenetic Stimulation

FIG. 7A is a flow chart of an experimental plan for chronic pain experimentation. FIG. 7B is an image of the CPP apparatus showing the dark (no treatment) chamber (left), the central chamber, and the lit (treatment-associated) chamber (right). FIG. 7C is a graphical comparison of chamber preferences of naïve unimplanted and implanted mice in CPP apparatus. There is no statistically significant change in preference between unimplanted mice and those with an ACC implant. N=5. FIG. 7D shows the percentage of post-conditioning time spent in the treatment-associated chamber increased. Despite the instinctive preference of rodents for darkened areas, the post-conditioning preference (while remaining in favor of the darkened room) increased towards the treatment-associated chamber. However, during live reinforcement, the preference for the treatment-associated chamber increased significantly. Avg±SEM. N=4, ** p<0.01.

### Example 7

### Long Term Safety of Optogenetic Implantation and Stimulation

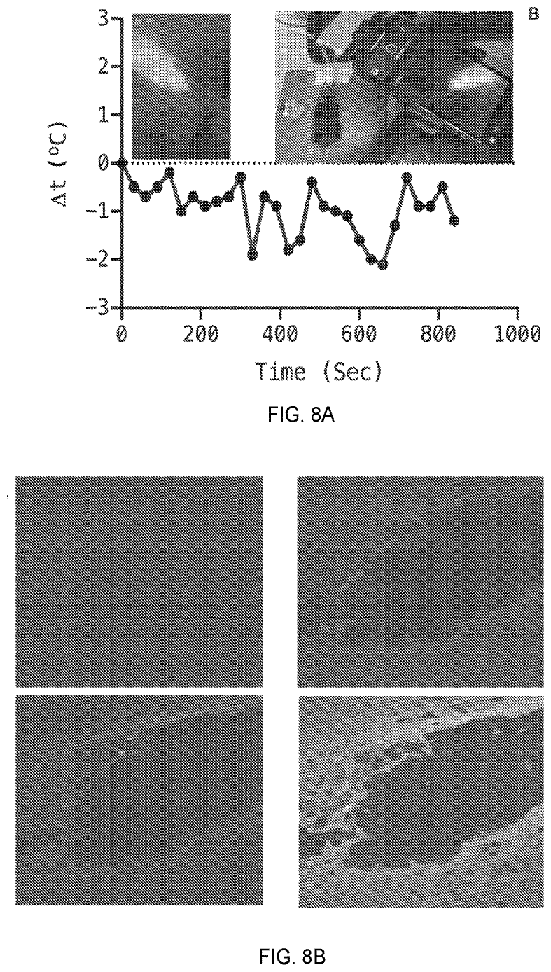

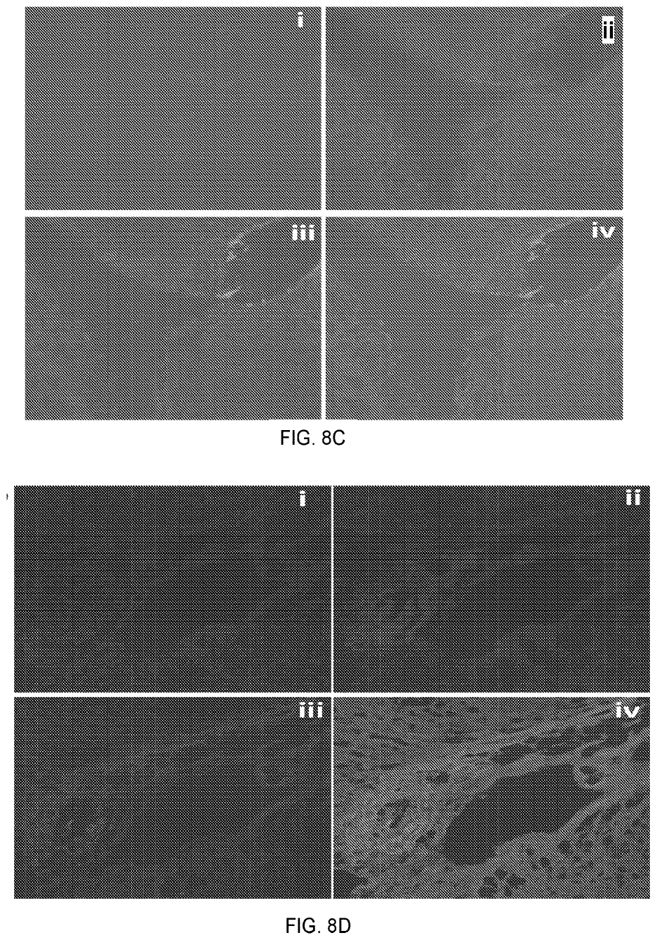

FIG. 8A shows the change in temperature of an irradiated spot in live brain measured by IR camera. Inset: Thermal image of the mouse under red-LED light stimulation. FIG. 8B is a series of images that shows no loss of viability of ACC-GABAergic neurons expressing MCO2-mCherry after chronic optogenetic stimulation with i) DAPI, (ii) MCO-mCherry, and (iii) Caspase-3. This is also depicted in an overlay (iv) of the images (i), (ii) and (iii). No apoptotic (Caspase +ve, green) cells were observed after eight sessions of one hour each (3 ms, 5 Hz, 0.4 mW). FIG. 8C shows the injection of AAV-MCOII led to MCO expression without causing inflammatory response with (i) DAPI, (ii) immunostained mCherry confirming MCO2-expression, (iii) CD45 immunostain (green) absent in ACC regions injected with 3 ml of AAV-MCO2 (8×10¹² vg/ml). This is also depicted in an overlay (iv) of (i), (ii) and (iii). FIG. 8D shows no immune cell response to implant in ACC transfected with MCO-mCherry. (i) DAPI, (ii) MCO-mCherry, (iii) Iba1 (marker for microglia/macrophages). This is also depicted in an overlay (iv) of the images (i), (ii) and (iii). Minimal Iba1 +ve (green) cells were observed in the vicinity of the implant.

The changes seen in mechanical allodynia reflected in the Von Frey assay also give indications of to the role of the ACC in pain. It is notable that while there is an immediate increase in the mechanical threshold, the significant reduction in mechanical allodynia is only apparent after 20 minutes of stimulation. Thus, it stands to reason that there is no strong direct inhibition of sensation occurring but rather some evolving modulation of how that input is interpreted and processed in the CNS. The comparable sensitivity of treated and unligated paw mechanical thresholds is also promising in that the goal is not to numb pain to the point of potential danger but to reduce chronic pain to manageable levels that allow a return to normal beneficial function.

The use of optogenetic stimulation allows effective pain modulation in a cell-specific and highly customizable manner. This is suitable to address the diversity of the pain experience. In our current research, we have seen that central methods allow recalcitrant pain syndromes of multiple etiologies and symptomologies to be addressed with a single implant. This is reflected in the reduction of reflexive pain responses in mice with optogenetics-based anterior cingulate cortex stimulation. The results show that the stimulation schedule (i.e., the frequency of stimulation sessions and the power of said sessions) correlates with the level of acute pain reduction as the intermittent treatment schedules showed higher average pain scores than constant stimulation. Moreover, the reduction of chronic neuropathic pain via optogenetic stimulation motivated an increase in the time spent in a normally aversive lit chamber. This supports the value of optogenetic methods as an alternative to traditional pharmacological methods.

### Example 8

### Effect of Delayed Optogenetic Stimulation on Pain Modulation

Even when optogenetic treatment (stimulation of MCO-transduced inhibitory neurons in ACC) is delayed, it shows statistically significant reduction of the pain score in the inflammatory phase of the formalin assay (FIG. 9).

FIGS. 9A and 9B show that inflammatory pain responses are reduced in even with delayed (starting 15 minutes into the experimental session) treatment with 5 Hz (5 ms pulses of 630 nm) optogenetic stimulation. FIG. 9A shows the results of formalin assay scores at ten-minute intervals comparing untreated baseline to treated experimental mice. The average of the cumulative pain scores in 5-minute intervals in the early (0-11 minutes) and late (20-41 minutes) phases of the experimental session in mice (FIG. 9B) with delayed treatment. Av.±SEM. N=7 at baseline and 4 in the treatment group, *p<0.05.

### Example 9

Persistence of Pain Modulation after Cessation of Optogenetic Stimulation

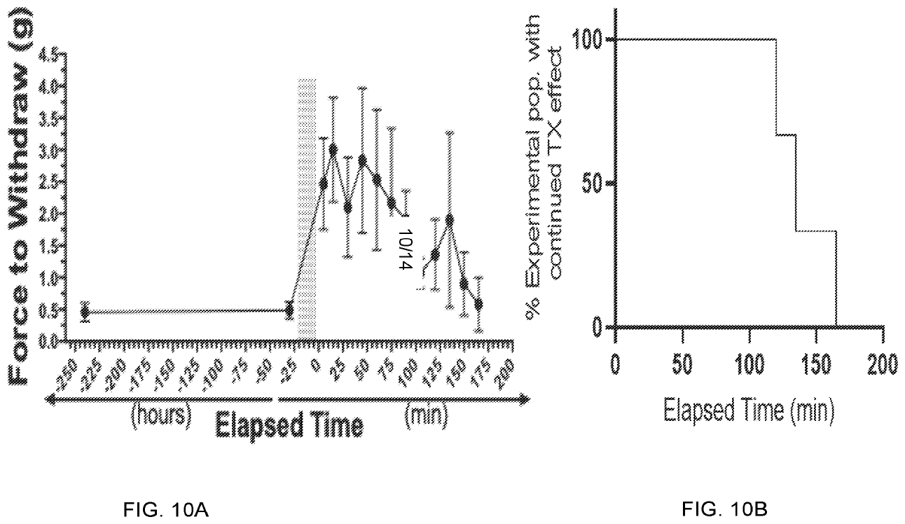

The analgesic effect persisted in the model of chronic pain (sciatic nerve ligation) even after cessation of optogenetic stimulation of the CNS. The typical mechanical hypersensitivity of the affected limb remains compensated long after the optogenetic stimulation has ended. The mechanical threshold remained elevated from the prestimulation baseline after a single 20-minute interval of optogenetic stimulation (FIG. 10A and FIG. 10B).

FIG. 10A and FIG. 10B show the persistence of the modulatory effect of MCO-based optogenetic stimulation of the CNS. FIG. 10A shows the force to cause hyperalgesic paw withdrawal (g) in experimental mice after cessation of optogenetic stimulation (20 min, marked by red bar). FIG. 10B shows the percentage of the experimental population that retained an increased mechanical sensitivity threshold after optogenetic stimulation. N=6, Av. SEM.

### Example 10

### No Change in Social Behavior Due to Opsin Expression and Optogenetic Stimulation of ACC

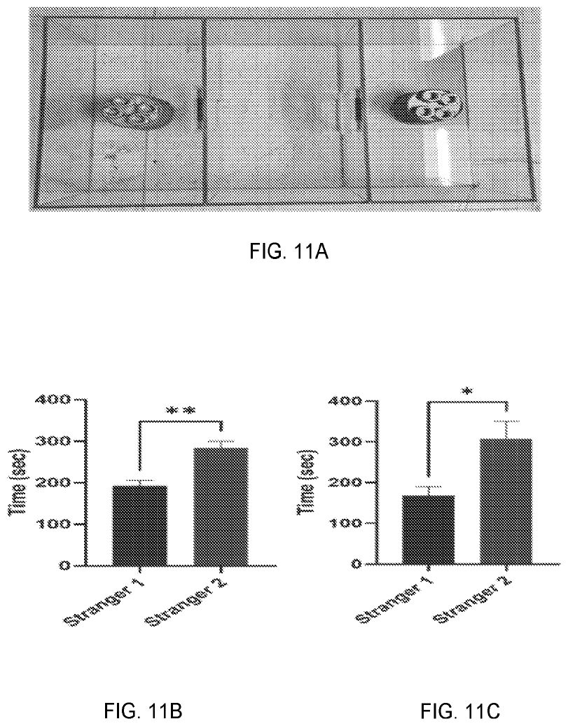

Implantation of the device in the CNS and MCO expression did not significantly affect the typical social novelty behavior of experimental mice. Both a wild type (non-implanted) and an implanted group (with MCO-transfected CNS) showed a similar affinity for novel social interactions (FIG. 11).

FIG. 11A-FIG. 11C show that MCO transfection of CNS-implantation does not impact social interaction. FIG. 11A is an image of a three-chamber social novelty apparatus. The preference for novel stimulation encourages a statistically significant difference between time spent with a previously introduced stranger and a completely new stranger in both (FIG. 11B) wild-type control and (FIG. 11C) mice with MCO-expression in CNS. N=4. Av.±SEM. ** p≤0.01, *p≤0.05.

### Example 11

### Pain Modulation by Optogenetic Stimulation of DRG, Sensitized by Virally-Delivered MCO

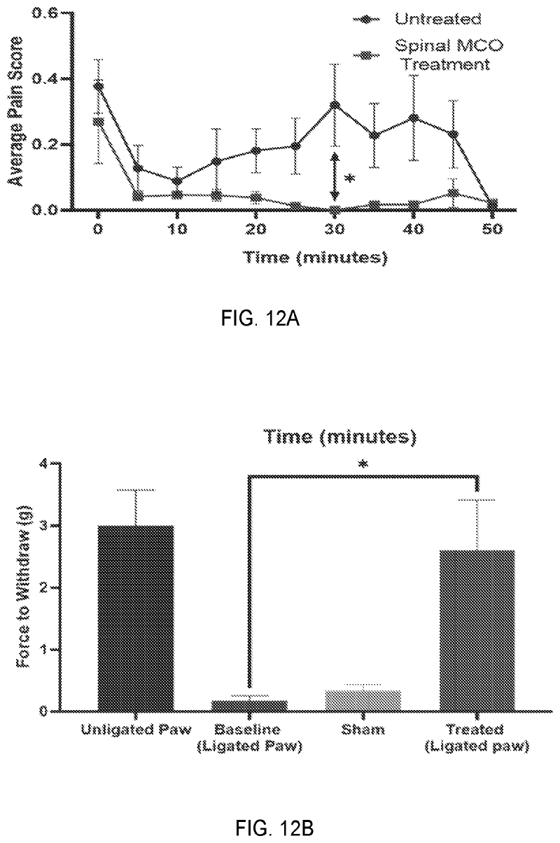

As shown in FIG. 12A, significant pain inhibition was observed with light stimulation of the spine (after intrathecal delivery of plasmids encoding MCO). The typical formalin test shows early (primary) and late (secondary) phase pain responses reflecting direct and inflammatory pain, respectively. Furthermore, we modeled chronic neuropathic pain using peripheral nerve injury models, and optogenetic stimulation compensated for the hypersensitivity seen in these models as reflected in a statistically significant increase in the minimum force needed to induce a paw withdrawal response in the affected limb (FIG. 12B).

FIG. 12A and FIG. 12B show that acute pain responses are reduced with continuous 5 Hz treatment (5 ms pulses at 630 nm). FIG. 12A shows pain responses as reflected in quantifiable behaviors during the Formalin assay at five-minute intervals with continuous optogenetic stimulation in the Spine reflect this change. FIG. 12B shows the minimum force needed to elicit hind paw withdrawal response to mechanical stimulation increased with spinal optogenetic stimulation. Avg±SEM. Untreated N=13 and Spinal treatment N=4. * p≤0.05.

### Example 12

### No Change in Social Behavior Due to Opsin Expression in DRG

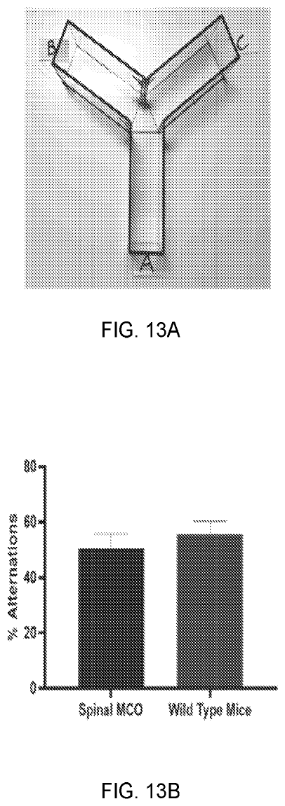

Spinal expression of MCO likewise had no significant effect on spontaneous alternation in a Y maze. Comparison of wild type to MCO-expressing mice (injected intrathecally with MCO-carrying-AAV) showed no significant difference in their typical exploration activity through a Y maze (FIG. 13).

FIG. 13A and FIG. 13B shows results of Y-maze Spontaneous Alternation. FIG. 13A is an image of an open Y-maze with labeled arms. FIG. 13B shows a comparison of spontaneous alternation between wild-type and MCO-expressing mice. N=4. Av.±SEM.

### Example 13

Inhibition of Pain Responses Persists after Premature Termination of Optogenetic Stimulation

Optogenetic stimulation of the Anterior Cingulate Cortex led to a significant reduction of the average pain responses even after termination. Comparison of wild type to MCO-expressing mice (injected with MCO-carrying-AAV in the ACC) showed a notable difference in their pain responses after cessation of optogenetic stimulation.

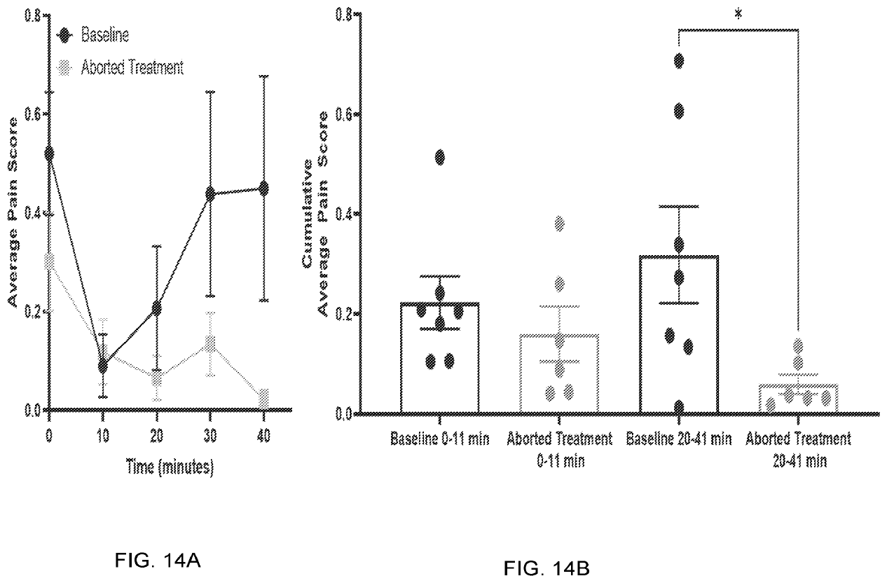

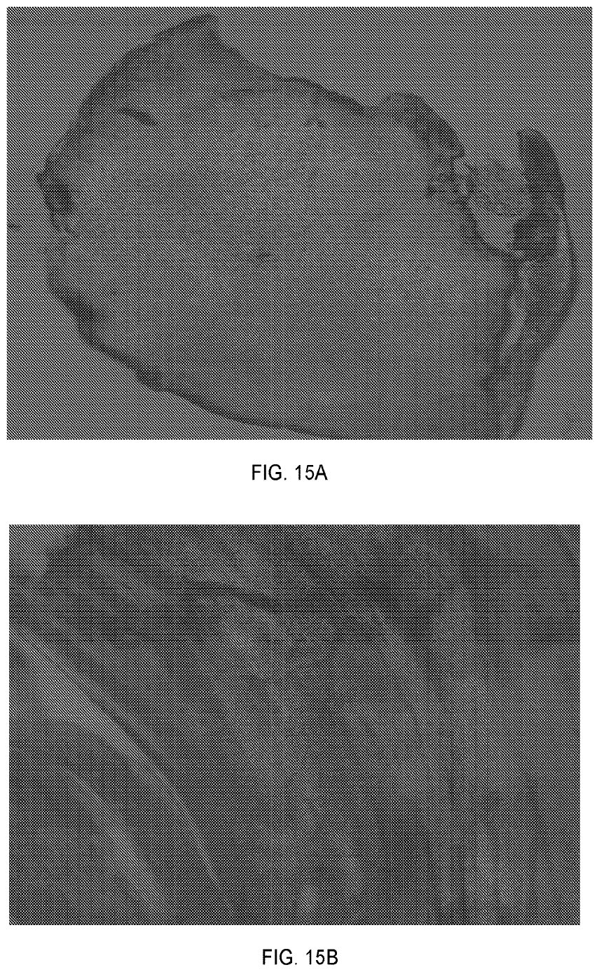

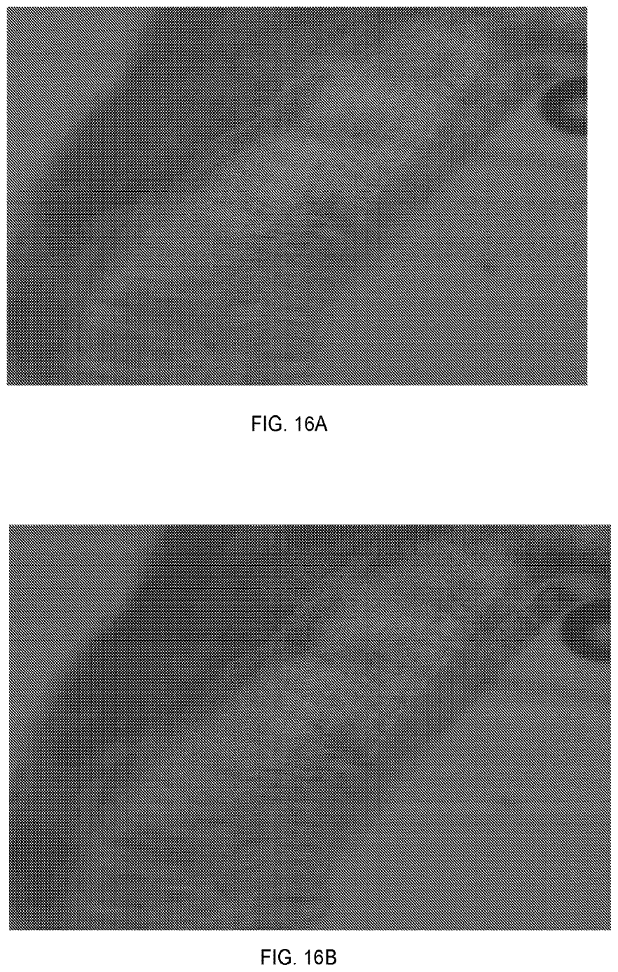

FIG. 14A and FIG. 14B shows that acute pain responses remain reduced after termination of optogenetic stimulation. Formalin assay scores at ten-minute intervals with 5 ms pulses (at 630 nm) comparing baseline to treated experimental mice A) with 5 Hz treatment for 20 minutes (aborted treatment). The average of the cumulative pain scores in five-minute intervals in the early (0-11 minutes) and late (20-41 minutes) phases of the experimental session in mice C) with aborted treatment. Av.±SEM. N=7 at baseline and 6 in the treatment group. * p<0.05

### Example 14

### Use of Optogenetic Stimulation to Treat Sciatica and Lower Back Pain

Targeting a central neurological structure with an integral role in pain perception bypasses many of the treatment shortcomings and challenges inherent in conventional approaches related to electrode implantation and non-specificity. Optogenetic modulation addresses these shortcomings by allowing cell-specific stimulation with low power. The illumination comes with a low power burden and can be tuned to respond to wavelengths that propagate better through tissues without damaging cells.

Sciatica refers to pain that travels along the path of the sciatic nerve. It is pain that starts along the sciatic nerve and spreads down the buttock and the back of the thigh. It is usually caused by a herniated (or bulging) disk in your spine that presses on your sciatic nerve. The pain can vary from a mild ache to a sharp, burning pain. Sometimes it feels like a jolt or electric shock.

In this example, a patient (i.e., a 50-year-old male) visits a healthcare provider complaining of persistent pain, tingling and muscle weakness that radiates from the lower back to the leg. The patient is otherwise in good health and wishes to avoid taking pain relievers. The healthcare professional recommends a treatment regime that includes optogenetic modulation of the CNS to reduce the sciatic pain.

Multi-Characteristic Opsin (MCO2) is packaged in an adenovirus, adeno-associated virus, or lentivirus vector, and is injected into neural cells (i.e., spinal cord, DRG, and/or ACC cell) of the patient. Two to four weeks after injection, the health care provider confirms expression in the targeted cells.

The patient also receives an implanted optical fiber that acts as a light source to deliver light to the targeted cells. In this example, the light source is activated via a wireless signal. Further, the light source is charged wirelessly (e.g., via inductive charging) thus avoiding the requirement of an internal or linked power source.

The light-emitting diode (LED) is controlled to generate pulses with a pulse width between 1 to 100 milliseconds, using a duty cycle between 1 to 100 percent. The LED is arranged so as to illuminate the target areas, wherein the tip is shaped flat or tapered so as to control the shape of the emanating light beam. The intensity of light emanating from the light source or waveguide coupled to the light source ranges between 1 mW/mm² to 100 mW/mm².

The light source generates pulses of light when triggered by the patient (or health care provider) or by a pre-set program. The light activates the transfected neural cells using visible light. Light-induced activation of multi-characteristic opsin (MCO) results in depolarization of only those cells that express MCO2.

Thereafter, the patient is evaluated for sciatic pain. The patient reports that almost all of the pain has subsided (approximately 90%). The patient is advised to continue pursuing a healthy lifestyle and activate the optical fiber for pain as needed.

The administration dose and frequency of the pharmaceutical compositions disclosed herein are determined by the type of active ingredient, together with various factors such as the disease to be treated, administration route, patient's age, gender, body weight, and disease severity. Moreover, the pharmaceutical compositions may be administered alone or in combination or coincident with other pharmaceutical formulations showing prophylactic or therapeutic efficacy.

In various embodiments, a formulation can include, without limitation, combinations of bioactive agents (such as viruses, proteins, antibodies, peptides, and the like as described herein) in the formulation. For example, a formulation as described herein can include a single bioactive agent for treatment of one or more conditions, including without limitation, disease. A formulation as described herein also can include, in an embodiment, without limitation, two or more different bioactive agents for a single or multiple conditions. Use of multiple bioactive agents in a formulation can be directed to, for example, the same or different indications. Similarly, in another embodiment, multiple bioactive agents can be used in a formulation to treat, for example, both a pathological condition and one or more side effects caused by the primary treatment. In a further embodiment, multiple bioactive agents also can be included, without limitation, in a formulation as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

Packaging and instruments for administration may be determined by a variety of considerations, such as, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto-injectors, safety syringes, injection pumps, infusion pumps, glass prefilled syringes, plastic prefilled syringes, and needle-free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, California, U.S.A. Administration by injection may be, without limitation intravenous, intramuscular, intraperitoneal, or subcutaneous, as appropriate. Administrations by non-injection route may be, without limitation, nasal, oral, ocular, dermal, or pulmonary, as appropriate.

In certain embodiments, kits can include one or more single or multi-chambered syringes (e.g., liquid syringes and lyosyringes) for administering one or more formulations described herein. In various embodiments, the kit can comprise formulation components for parenteral, subcutaneous, intramuscular, or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The formulations as described herein can be used in the manufacture of medicaments and for the treatment of humans and other animals by administration in accordance with conventional procedures.

Also provided herein are combinatorial methods for developing suitable virus formulations using combinations of amino acids. These methods are effective for developing stable liquid or lyophilized formulations, particularly pharmaceutical virus formulations.

Compositions in accordance with embodiments described herein have desirable properties, such as desirable solubility, viscosity, syringeability and stability. Lyophilates in accordance with embodiments described herein have desirable properties, as well, such as desirable recovery, stability, and reconstitution.

In an embodiment, the method provides 1, 5, 10, 15, 20, 30, 40, 50, 60, 70, 80, 90 or greater percent reduction in the pain (e.g., neuropathic or chronic pain).

In an embodiment, the period of a therapeutic method described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutic method described herein reduces signs/symptoms in an individual suffering from a pain by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutic method disclosed herein reduces signs/symptoms such as pain by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutic method disclosed herein reduces signs/symptoms such as pain by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects, a therapeutic method disclosed herein reduces a level of pain in an individual (e.g., neuropathic, nociceptive or chronic pain) by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutic method disclosed herein reduces pain by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutic method disclosed herein reduces pain by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50% (as compared to an untreated subject).

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

In closing, it is to be understood that although aspects of the specification are highlighted by referring to specific embodiments, one skilled in the art will appreciate that these embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

| Sequence Listings: | |
|---|---|
| MCO2 from PAAC-Gad67MCO2-mcherry | |
| SEQ ID NO: 1 | |
| ATGGATTACG GTGGTGCACT TTCGGCCGTG GGGCGTGAGC TGCTATTCGT GACGAACCCG | 60 |
| GTAGTCGTCA ACGGCTCAGT CCTTGTCCCC GAAGACCAAT GCTATTGTGC AGGATGGATA | 120 |
| GAAAGCAGAG GCACGAACGG CGCGCAAACG GCATCTAATG TGCTACAGTG GTTAGCTGCA | 180 |
| GGCTTTTCGA TACTTCTATT GATGTTTTAT GCATACCAAA CATGGAAATC TACATGCGGC | 240 |
| TGGGAAGAGA TTTATGTCTG CGCAATCGAA ATGGTGAAGG TTATCCTGGA GTTCTTCTTT | 300 |
| GAATTCAAAA ACCCTTCAAT GTTGTATCTC GCAACAGGGC ATAGAGTCCA GTGGTTAAGG | 360 |
| TACGCAGAGT GGCTCTTGAC TTGTCCTGTT ATACTTATAC ACCTGAGCAA TCTCACTGGC | 420 |
| TTAAGTAATG ATTATAGCCG ACGGACAATG GGCTTGTTAG TTTCAGACAT CGGAACTATC | 480 |
| GTATGGGGGG CCACAAGCGC TATGGCCACT GGCTACGTTA AGGTAATTTT TTTTTGCCTG | 540 |
| GGATTGTGTT ACGGCGCAAA CACATTTTTC CATGCAGCAA AAGCTTATAT TGAAGGTTAC | 600 |
| CATACAGTTC CCAAAGGACG GTGCAGACAA GTCGTCACAG GAATGGCGTG GCTGTTCTTT | 660 |
| GTAAGTTGGG GTATGTTCCC CATATTATTC ATCCTTGGCC CAGAGGGCTT TGGAGTGCTG | 720 |
| TCTGTCTATG GATCTACAGT AGGTCACACT ATCATTGATT TGATGAGCAA AAACTGCTGG | 780 |
| GGCCTTCTAG GACACTACTT ACGCGTCCTG ATTCATGAAC ACATTCTCAT TCACGGAGAC | 840 |
| ATACGTAAGA CGACCAAGCT AAATATCGGA GGAACGGAAA TCGAGGTTGA AACACTTGTA | 900 |
| GAGGATGAGG CTGAAGCTGG AGCTATGGCT GAGCTGATCA GCAGCGCCAC CAGATCTCTG | 960 |
| TTTGCCGCCG GAGGCATCAA CCCTTGGCCT AACCCCTACC ACCACGAGGA CATGGGCTGT | 1020 |
| GGAGGAATGA CACCTACAGG CGAGTGCTTC AGCACCGAGT GGTGGTGTGA CCCTTCTTAC | 1080 |
| GGACTGAGCG ACGCCGGATA CGGATATTGC TTCGTGGAGG CCACAGGCGG CTACCTGGTC | 1140 |
| GTGGGAGTGG AGAAGAAGCA GGCTTGGCTG CACAGCAGAG GCACACCAGG AGAAAAGATC | 1200 |
| GGCGCCCAGG TCTGCCAGTG GATTGCTTTC AGCATCGCCA TCGCCCTGCT GACATTCTAC | 1260 |
| GGCTTCAGCG CCTGGAAGGC CACTTGCGGT TGGGAGGAGG TCTACGTCTG TTGCGTCGAG | 1320 |
| GTGCTGTTCG TGACCCTGGA GATCTTCAAG GAGTTCAGCA GCCCCGCCAC AGTGTACCTG | 1380 |
| TCTACCGGCA ACCACGCCTA TTGCCTGCGC TACTTCGAGT GGCTGCTGTC TTGCCCCGTG | 1440 |
| ATCCTGATCA GACTGAGCAA CCTGAGCGGC CTGAAGAACG ACTACAGCAA GCGGACCATG | 1500 |
| GGCCTGATCG TGTCTTGCGT GGGAATGATC GTGTTCGGCA TGGCCGCAGG ACTGGCTACC | 1560 |
| GATTGGCTCA AGTGGCTGCT GTATATCGTG TCTTGCATCT ACGGCGGCTA CATGTACTTC | 1620 |
| CAGGCCGCCA AGTGCTACGT GGAAGCCAAC CACAGCGTGC CTAAAGGCCA TTGCCGCATG | 1680 |
| GTCGTGAAGC TGATGGCCTA CGCTTACTTC GCCTCTTGGG GCAGCTACCC AATCCTCTGG | 1740 |
| GCAGTGGGAC CAGAAGGACT GCTGAAGCTG AGCCCTTACG CCAACAGCAT CGGCCACAGC | 1800 |
| ATCTGCGACA TCATCGCCAA GGAGTTTTGG ACCTTCCTGG CCCACCACCT GAGGATCAAG | 1860 |
| ATCCACGAGC ACATCCTGAT CCACGGCGAC ATCCGGAAGA CCACCAAGAT GGAGATCGGA | 1920 |
| GGCGAGGAGG TGGAAGTGGA AGAGTTCGTG GAGGAGGAGG ACGAGGACAC AGTGGTGAGC | 1980 |
| AAGGGCGAGG AGGATAA | 1997 |

1. An optogenetic stimulation apparatus for use in a method of inhibiting pain by targeting inhibitory cells with an excitatory light-sensitive protein in a subject suffering from pain, the apparatus comprising an optogenetic stimulation device for emitting light and controlling the emission of the light, and a recombinant nucleic acid encoding an excitatory light-sensitive protein, wherein the method comprising steps of:
a) expressing the recombinant nucleic acid encoding the excitatory light-sensitive protein in inhibitory neural cells of the subject, and
b) controlling expression of the excitatory light-sensitive protein in GABAergic inhibitory cells by use of a GAD promoter to modulate neural activity of the inhibitory cells with light generated by the optogenetic stimulation device,
wherein the modulation reduces the duration and/or intensity of the pain.

2-61. (canceled)

62. The optogenetic stimulation apparatus of claim 1, wherein the inhibitory neural cells are spinal cord cells, dorsal root ganglioa (DRG) cells, and/or anterior cingulate cortex (ACC) cells.

63. The optogenetic stimulation apparatus of claim 1, wherein the excitatory light-sensitive protein is a multi-characteristic opsin (MCO).

64. The optogenetic stimulation apparatus of claim 63, wherein the multi-characteristic opsin (MCO) has at least 80% identity to SEQ ID NO. 1.

65. The optogenetic stimulation apparatus of claim 1, wherein the pain is one or more of neuropathic pain, inflammatory pain, headache pain, somatic pain visceral pain and referred pain.

66. The optogenetic stimulation apparatus of claim 65, wherein the neuropathic pain is chronic neuropathic pain and/or caused by one or more of a traumatic insult, a spinal cord injury, a limb amputation, a contusion, an inflammation or a surgical procedure, an ischemic event, an infectious agent, exposure to a toxic agent or a disease.

67. The optogenetic stimulation apparatus of claim 1, comprising a viral vector or non-viral vector for use in the step of expressing a recombinant nucleic acid encoding a light-sensitive protein in the inhibitory neural cells of the subject.

68. The optogenetic stimulation apparatus of claim 1, comprising one or more optical fiber and wherein the method further comprising a step of implanting the one or more optical fiber into the subject to control light activation of the light-sensitive protein, optionally wherein the one or more optical fiber is operable to be activated from a wireless signal, and optionally wherein the one or more optical fiber is configured to be implanted charged, or be powered wirelessly.

69. The optogenetic stimulation apparatus of claim 68, wherein the light is a low-power red light, optionally wherein the light has a wavelength of about 635 nm.

70. The optogenetic stimulation apparatus of claim 1 for use in a method of inhibiting neuropathic pain in a subject without affecting nociceptive pain,
wherein light activation of the excitatory light-sensitive protein reduces the duration and/or intensity of the neuropathic pain.

71. The optogenetic stimulation apparatus of claim 1 for use in a method of treating an ailment in a subject in need thereof, wherein the modulation adjusts the excitation to inhibition (E/I) balance of the inhibitory neural cells of the subject.

72. The optogenetic stimulation apparatus of claim 62, wherein the ailment is a neurological disorder selected from one or more of the fibromyalgia, rheumatoid arthritis, osteoarthritis, chronic arthropathy, spinal nerve compression syndromes associated with neoplasia and/or disc herniation, chronic back pain, chronic joint pain of any etiology associated with inflammation and/or structural joint abnormalities, post-herpetic neuralgia, trigeminal neuralgia, chronic metabolic neuropathy associated with chronic pain, migraine, inflammatory pain, post-surgical pain syndromes including phantom limb pain, post-traumatic stress disorder (PTSD), irritable bowel syndrome, autonomic neuropathies, arachnoiditis, chronic regional pain syndrome, vulvodynia, and chronic pain syndrome associated with activation of central sensitization pathways, visual impairment, drug addiction, a psychological disorder, and a movement disorder.

73. The optogenetic stimulation apparatus of claim 62, wherein the persistence of therapy is maintained by modulating light parameters, optionally wherein the light parameters are at least one selected from intensity, duty cycle, and periods.

74. The optogenetic stimulation apparatus of claim 1 for use in a method of reducing pain in a subject, wherein the optogenetic stimulation device is wireless, and the method comprising steps of:
a) transfecting the inhibitory neural cells of the subject with a virus to express the excitatory light-sensitive protein;
b) implanting the wireless optogenetic stimulation device at or near the inhibitory neural cells, the spinal cord, ACC or DRG of the subject; and
c) activating the excitatory light-sensitive protein in specific inhibitory neural cells in the spinal cord, ACC or DRG with light emitted from the optogenetic stimulation device to reduce pain.

75. The optogenetic stimulation apparatus of claim 65, wherein the light is emitted in pulses with a width from 1 to 100 milliseconds and a duty cycle from 1 to 100 percent, and tuned to a light intensity up to 100 mW/mm².

76. A method of inhibiting pain by targeting inhibitory cells with an excitatory light-sensitive protein in a subject suffering from pain, the method comprising steps of:
a) expressing a recombinant nucleic acid encoding the excitatory light-sensitive protein in inhibitory neural cells of the subject, and
b) controlling expression of the excitatory light-sensitive protein in GABAergic inhibitory cells by use of a GAD promoter to modulate neural activity of the inhibitory cells with light,
wherein the modulation reduces the duration and/or intensity of the pain, and using an LED light source for generating the light, optionally comprising fiber optic(s).

77. The method of claim 67, wherein the inhibitory neural cells are spinal cord cells, dorsal root ganglioa (DRG) cells, and/or anterior cingulate cortex (ACC) cells.

78. The method of claim 67, wherein the excitatory light-sensitive protein is a multi-characteristic opsin (MCO), optionally wherein the multi-characteristic opsin (MCO) has at least 80% identity to SEQ ID NO. 1.

79. The method of claim 67, wherein the pain is one or more of neuropathic pain, inflammatory pain, headache pain, somatic pain visceral pain and referred pain, optionally wherein the neuropathic pain is chronic neuropathic pain and/or caused by one or more of a traumatic insult, a spinal cord injury, a limb amputation, a contusion, an inflammation or a surgical procedure, an ischemic event, an infectious agent, exposure to a toxic agent or a disease.

80. The method of claim 67, wherein a light source for generating the light is implanted or transdermal, and the light is a low-power red light, optionally wherein the light has a wavelength of about 635 nm.


SEQUENCE LISTING (1 sequence)

SEQ ID NO: 1 - DNA, 1997 bp, synthetic construct
  misc_feature 1..1997: note = MCO2 from PAAC-Gad67MCO2-mcherry
  source 1..1997: mol_type = other DNA
atggattacggtggtgcactttcggccgtggggcgtgagctgctattcgtgacgaacccggtagtcgtcaacggctcagtccttgtccccgaagaccaatgctattgtgcaggatggatagaaagcagaggcacgaacggcgcgcaaacggcatctaatgtgctacagtggttagctgcaggcttttcgatacttctattgatgttttatgcataccaaacatggaaatctacatgcggctgggaagagatttatgtctgcgcaatcgaaatggtgaaggttatcctggagttcttctttgaattcaaaaacccttcaatgttgtatctcgcaacagggcatagagtccagtggttaaggtacgcagagtggctcttgacttgtcctgttatacttatacacctgagcaatctcactggcttaagtaatgattatagccgacggacaatgggcttgttagtttcagacatcggaactatcgtatggggggccacaagcgctatggccactggctacgttaaggtaatttttttttgcctgggattgtgttacggcgcaaacacatttttccatgcagcaaaagcttatattgaaggttaccatacagttcccaaaggacggtgcagacaagtcgtcacaggaatggcgtggctgttctttgtaagttggggtatgttccccatattattcatccttggcccagagggctttggagtgctgtctgtctatggatctacagtaggtcacactatcattgatttgatgagcaaaaactgctggggccttctaggacactacttacgcgtcctgattcatgaacacattctcattcacggagacatacgtaagacgaccaagctaaatatcggaggaacggaaatcgaggttgaaacacttgtagaggatgaggctgaagctggagctatggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgcttacttcgcctcttggggcagctacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtggtgagcaagggcgaggaggataa